(19) United States
(12) Patent Application Publication
(10) Pub. No.: US 2025/0008963 A1
(43) Pub. Date: Jan. 9, 2025

(54) Microbial Compositions and Methods for Improving Traits of Agronomic Importance in Plants

(71) Applicant: RAISON, LLP, Sioux Falls, SD (US)

(72) Inventor: Tony Hagen, Sioux Falls, SD (US)

(21) Appl. No.: 18/703,142

(22) PCT Filed: Oct. 19, 2022
(86) PCT No.: PCT/US2022/047136

§ 371 Date: Apr. 19, 2024

Related U.S. Application Data
(60) Provisional application No. 63/257,517, filed on Oct. 19, 2021.

(51) Int. Cl. A01N 63/27; A01P 21/00; C12N 1/20; C12R 1/145; C12R 1/38

(52) U.S. Cl. CPC A01N 63/27; A01P 21/00; C12N 1/20; C12R 2001/145; C12R 2001/38

(57) ABSTRACT

Disclosed herein are compositions and using said compositions in methods for improving agronomic traits in plants. Also disclosed herein are compositions and using said compositions in methods for increasing the sugar content and sugar holding capacity of grapes and melons.

### CROSS REFERENCE TO RELATED APPLICATIONS

This application claims the benefit of U.S. Provisional Application No. 63/257,517, filed Oct. 19, 2021. The content of this earlier filed application is hereby incorporated by reference herein in its entirety.

### FIELD

The present disclosure relates to compositions and methods for improving cultivation of plants, and, in particular, improving agronomic traits in plants. The disclosure provides a microbial ensemble, and further relates to methods of using the microbial ensemble.

### SUMMARY

The present disclosure relates to compositions comprising a Pseudomonas spp. and a Clostridium spp., and methods for using said compositions to improving agronomic traits in plants.

Disclosed herein are methods of improving a trait of agronomic importance in a plant, the methods comprising: (a) contacting a plant element of the plant with a composition comprising a first bacterial strain comprising Clostridium spp. with a 16S nucleic acid sequence that is at least about 97% identical to any one of the Clostridium spp. listed in Table 1 or Table 2 and a second bacterial strain comprising aquatic Pseudomonas spp. with a 16S nucleic acid sequence that is at least about 97% identical to any one of the Pseudomonas spp. listed in Table 1 or Table 2; and (b) growing the plant element comprising the composition comprising the first bacterial strain and the second bacterial strain into the plant, wherein the plant grown from the contacted plant element has an improvement in the trait of agronomic importance as compared to a plant not contacted with the composition.

Disclosed herein are methods of improving a trait of agronomic importance in a plant, the methods comprising: (a) contacting a plant element of the plant with a composition comprising one or more of the microbes listed in Table 1, Table 2, Table 3 or Table 4; and (b) growing the plant element comprising the composition comprising one or more of the microbes listed in Table 1, Table 2, Table 3 or Table 4 into the plant, wherein the plant grown from the contacted plant element has an improvement in the trait of agronomic importance as compared to a plant not contacted with the composition.

Disclosed herein are methods of improving a trait of agronomic importance in a plant, the methods comprising: growing the plant from a plant reproductive element that has been contacted with a composition comprising a first bacterial strain comprising Clostridium spp. with a 16S nucleic acid sequence that is at least about 97% identical to any one of the Clostridium spp. listed in Table 1 or Table 2 and a second bacterial strain comprising aquatic Pseudomonas spp. with a 16S nucleic acid sequence that is at least about 97% identical to any one of the Pseudomonas spp. listed in Table 1 or Table 2; wherein the plant grown from the contacted reproductive element comprises the composition comprising the first bacterial strain and the second bacterial strain, and wherein the plant grown from the contacted plant reproductive element has an improvement in a trait of agronomic importance as compared to an isoline plant grown from a plant reproductive element not contacted with the composition.

Disclosed herein are methods of improving a trait of agronomic importance in a plant, the methods comprising growing the plant from a plant reproductive element that has been contacted with a composition comprising one or more of the microbes listed in Table 1. Table 2. Table 3 or Table 4; wherein the plant grown from the contacted reproductive element comprises the composition comprising the one or more microbes, and wherein the plant grown from the contacted plant reproductive element has an improvement in a trait of agronomic importance as compared to an isoline plant grown from a plant reproductive element not contacted with the composition.

Disclosed herein are methods of increasing the sugar content of a grape or melon, the methods comprising: contacting an effective amount of a composition comprising a first bacterial strain comprising Clostridium spp. with a 16S nucleic acid sequence that is at least about 97% identical to any one of the Clostridium spp. listed in Table 1 or Table 2 and a second bacterial strain comprising aquatic Pseudomonas spp. with a 16S nucleic acid sequence that is at least about 97% identical to any one of the Pseudomonas spp. listed in Table 1 or Table 2 with an external surface of the grape or melon wherein the sugar content of the grape or melon contacted with the composition has an increased sugar content as compared to a plant not contacted with the composition.

Disclosed herein are methods of increasing the sugar content of a grape or melon, the methods comprising: contacting an effective amount of a composition comprising one or more of the microbes listed in Table 1, Table 2, Table 3 or Table 4 with an external surface of the grape or melon wherein the sugar content of the grape or melon contacted with the composition has an increased sugar content as compared to a plant not contacted with the composition.

Disclosed herein are methods of increasing the sugar holding capacity of a grape or melon, the methods comprising contacting an effective amount of a composition comprising a first bacterial strain comprising Clostridium spp. with a 16S nucleic acid sequence that is at least about 97% identical to any one of the Clostridium spp. listed in Table 1 or Table 2 and a second bacterial strain comprising aquatic Pseudomonas spp. with a 16S nucleic acid sequence that is at least about 97% identical to any one of the Pseudomonas spp. listed in Table 1 or Table 2 with an external surface of the grape or melon wherein the sugar holding capacity of the grape or melon contacted with the composition has an increased sugar content as compared to a plant not contacted with the composition.

Disclosed herein are methods of increasing the sugar holding capacity of a grape or melon, the methods comprising contacting an effective amount of a composition comprising one or more of the microbes listed in Table 1, Table 2, Table 3 or Table 4 with an external surface of the grape or melon wherein the sugar holding capacity of the grape or melon the contacted with the composition has an increased sugar content as compared to a plant not contacted with the composition.

Disclosed herein are methods of increasing the amount of atmospheric derived nitrogen in a plant, the methods comprising: exposing the plant to a composition comprising a first bacterial strain comprising Clostridium spp. with a 16S nucleic acid sequence that is at least about 97% identical to any one of the Clostridium spp. listed in Table 1 or Table 2 and a second bacterial strain comprising aquatic Pseudomonas spp. with a 16S nucleic acid sequence that is at least about 97% identical to any one of the Pseudomonas spp. listed in Table 1 or Table 2, and exposing the plant to one or more nitrogen-fixing bacteria thereby increasing an amount of atmospheric derived nitrogen within the plant that is exposed to the composition and the one or more nitrogen-fixing bacteria relative to a plant that is not exposed to the composition.

Disclosed herein are methods of increasing the amount of atmospheric derived nitrogen in a plant, the methods comprising: exposing the plant to a composition comprising one or more of the microbes listed in Table 1, Table 2, Table 3 or Table 4, and exposing the plant to one or more nitrogen-fixing bacteria thereby increasing an amount of atmospheric derived nitrogen within the plant that is exposed to the composition and the one or more nitrogen-fixing bacteria relative to a plant that is not exposed to the composition.

Disclosed herein are methods of increasing nitrogen production in a plant, the methods comprising: (a) contacting a plant element of the plant with a composition comprising a first bacterial strain comprising Clostridium spp. with a 16S nucleic acid sequence that is at least about 97% identical to any one of the Clostridium spp. listed in Table 1 or Table 2 and a second bacterial strain comprising aquatic Pseudomonas spp. with a 16S nucleic acid sequence that is at least about 97% identical to any one of the Pseudomonas spp. listed in Table 1 or Table 2 and (b) growing the plant element comprising the first bacterial strain and the second bacterial strain into the plant, wherein the plant grown from the contacted plant element produces more nitrogen as compared to a plant not contacted with the composition.

Disclosed herein are methods of increasing nitrogen production in a plant, the methods comprising: (a) contacting a plant element of the plant with a composition comprising one or more of the microbes listed in Table 1, Table 2, Table 3 or Table 4 and (b) growing the plant element comprising the one or more of the microbes into the plant, wherein the plant grown from the contacted plant element produces more nitrogen as compared to a plant not contacted with the composition.

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “comprising” can include the aspects “consisting of” and “consisting essentially of.”

The term “plant” is used herein to include any plant, tissues or organs (e.g., plant parts). Plant parts include, but are not limited to, cells, stems, roots, flowers, ovules, stamens, seeds, leaves, that can be cultured into a whole plant. A plant cell is a cell of a plant, either taken directly from a seed or plant, or derived through culture from a cell taken from a plant.

As used herein, the term “plant” further includes the whole plant or any parts or derivatives thereof, such as plant cells, plant protoplasts, plant cell tissue cultures from which plants can be regenerated, plant calli, embryos, pollen, ovules, fruit, flowers, leaves, seeds, roots, root tips and the like.

The exposed plants can be further assessed to isolate polynucleotides, amino acid sequences and/or genetic markers that are associated with, linked to, the desired trait. Further assessments include, but are not limited to, isolating polynucleotides, nucleic acids, or amino acids sequences from the exposed plant, carrying out an assay of the isolated polynucleotides or nucleic acids, for example, to detect one or more biological or molecular markers associated with one or more agronomic characteristics or traits, including but not limited to, increased sugar content. The information gleaned from such methods can be used, for example, in a breeding program.

As used herein the terms “microorganism” or “microbe” are used interchangeably and include, but are not limited to, the two prokaryotic domains, Bacteria and Archaea, eukaryotic fungi and protozoa, as well as viruses. In some aspects, the disclosure refers to the “microbes” of Table 1, Table 2, and/or Table 3 or the “microbes” incorporated by reference. This characterization can refer to not only the predicted taxonomic microbial identifiers of the Tables, but also the identified strains of the microbes listed in the Tables.

The term “microbial consortia” or “microbial consortium” refers to a subset of a microbial community of individual microbial species, or strains of a species, which can be described as carrying out a common function, or can be described as participating in, or leading to, or correlating with, a recognizable parameter or plant phenotypic trait. The community may comprise two or more species, or strains of a species, of microbes. In some instances, the microbes coexist within the community symbiotically.

The term “microbial community” means a group of microbes comprising two or more species or strains. Unlike microbial ensemble, a microbial community does not have to be carrying out a common function, or does not have to be participating in, or leading to, or correlating with, a recognizable parameter, such as a phenotypic trait of interest (e.g., increased sugar content of a grape or melon).

As used herein, “isolate,” “isolated,” “isolated microbe,” and like terms, are intended to mean that the one or more microorganisms has been separated from at least one of the materials with which it is associated in a particular environment (for example soil, water, animal tissue).

Thus, an “isolated microbe” does not exist in its naturally occurring environment; rather, it is through the various techniques described herein that the microbe has been removed from its natural setting and placed into a non-naturally occurring state of existence. Thus, the isolated strain or isolated microbe may exist as, for example, a biologically pure culture, or as spores (or other forms of the strain) in association with an acceptable carrier.

As used herein, “spore” or “spores” refer to structures produced by bacteria and fungi that are adapted for survival and dispersal. Spores are generally characterized as dormant structures; however, spores are capable of differentiation through the process of germination. Germination is the differentiation of spores into vegetative cells that are capable of metabolic activity, growth, and reproduction. The germination of a single spore results in a single fungal or bacterial vegetative cell. Fungal spores are units of asexual reproduction, and in some cases are necessary structures in fungal life cycles. Bacterial spores are structures for surviving conditions that may ordinarily be nonconductive to the survival or growth of vegetative cells.

As used herein, “microbial composition” refers to a composition comprising one or more microbes of the present disclosure, wherein a microbial composition, in some aspects, is administered to animals of the present disclosure.

As used herein, “carrier”, “acceptable carrier”, or “pharmaceutical carrier” refers to a diluent, adjuvant, excipient, or vehicle with which the compound is administered. Such carriers can be sterile liquids, such as water and oils, including those of petroleum, animal, vegetable, or synthetic origin; such as peanut oil, soybean oil, mineral oil, sesame oil, and the like. Water or aqueous solution saline solutions and aqueous dextrose and glycerol solutions are preferably employed as carriers, in some embodiments as injectable solutions. In some embodiments, gelling agents are employed as carriers. Alternatively, the carrier can be a solid dosage form carrier, including but not limited to one or more of a binder (for compressed pills), a glidant, an encapsulating agent, a flavorant, and a colorant. The choice of carrier can be selected with regard to the intended route of administration and standard pharmaceutical practice. See Hardee and Baggo (1998. Development and Formulation of Veterinary Dosage Forms. 2nd Ed. CRC Press. 504 pg.); E. W. Martin (1970. Remington's Pharmaceutical Sciences. 17th Ed. Mack Pub. Co.); and Blaser et al. (US Publication US20110280840A1).

In some aspects, carriers may be granular in structure, such as sand or sand particles. In some aspects, the carriers may be dry, as opposed to a moist or wet carrier. In some aspects, carriers can be nutritive substances and/or prebiotic substances selected from fructo-oligosaccharides, inulins, isomalto-oligosaccharides, lactitol, lactosucruse, lactulose, pyrodextrines, soy oligosaccharides, transgalacto-oligosaccharides, xylo-oligosaccharides, trace minerals, and vitamins. In some aspects, carriers can be in solid or liquid form. In some aspects, carriers can be zeolites, calcium carbonate, magnesium carbonate, silicon dioxide, ground corn, trehalose, chitosan, shellac, albumin, starch, skim-milk powder, sweet-whey powder, maltodextrin, lactose, and inulin. In some aspects, a carrier is water or physiological saline.

The term “bioensemble,” “microbial ensemble,” or “synthetic ensemble” refers to a composition comprising one or more active microbes identified by methods, systems, and/or apparatuses of the present disclosure and that do not naturally exist in a naturally occurring environment and/or at ratios or amounts that do not exist in nature. A bioensemble is a subset of a microbial community of individual microbial species, or strains of a species, which can be described as carrying out a common function, or can be described as participating in, or leading to, or correlating with, a recognizable parameter, such as a phenotypic trait of interest (e.g. increased feed efficiency in feedlot cattle). The bioensemble may comprise two or more species, or strains of a species, of microbes. In some instances, the microbes coexist within the community symbiotically.

As used herein, “microbiome” refers to the collection of microorganisms that inhabit the digestive tract or gastrointestinal tract of an animal (including the rumen if said animal is a ruminant) and the microorganism's physical environment (i.e. the microbiome has a biotic and physical component). The microbiome is fluid and may be modulated by numerous naturally occurring and artificial conditions (e.g., change in diet, disease, antimicrobial agents, influx of additional microorganisms, etc.). The modulation of the microbiome of a rumen that can be achieved via administration of the compositions of the disclosure, can take the form of: (a) increasing or decreasing a particular Family, Genus, Species, or functional grouping of microbe (i.e., alteration of the biotic component of the rumen microbiome) and/or (b) increasing or decreasing volatile fatty acids in the rumen, increasing or decreasing rumen pH, increasing or decreasing any other physical parameter important for rumen health (i.e., alteration of the abiotic component of the rumen microbiome).

The term “growth medium” as used herein, is any medium which is suitable to support growth of a microbe. By way of example, the media may be natural or artificial including gastrin supplemental agar, LB media, blood serum, and tissue culture gels. It should be appreciated that the media may be used alone or in combination with one or more other media. It may also be used with or without the addition of exogenous nutrients.

The term “relative abundance” as used herein, is the number or percentage of a microbe present in the gastrointestinal tract or other organ system, relative to the number or percentage of total microbes present in said tract or organ system. The relative abundance may also be determined for particular types of microbes such as bacteria, fungi, viruses, and/or protozoa, relative to the total number or percentage of bacteria, fungi, viruses, and/or protozoa present. In one embodiment, relative abundance is determined by PCR. In another embodiment, relative abundance is determined by colony forming unit assays (cfu) or plaque forming unit assays (pfu) performed on samples from the gastrointestinal tract or other organ system of interest.

The medium may be amended or enriched with additional compounds or components, for example, a component which may assist in the interaction and/or selection of specific groups of microorganisms. For example, antibiotics (such as penicillin) or sterilants (for example, quaternary ammonium salts and oxidizing agents) could be present and/or the physical conditions (such as salinity, nutrients (for example organic and inorganic minerals (such as phosphorus, nitrogenous salts, ammonia, potassium and micronutrients such as cobalt and magnesium), pH, and/or temperature), methionine, prebiotics, ionophores, and beta glucans could be amended.

As used herein, “improved” should be taken broadly to encompass improvement of a characteristic of interest, as compared to a control group, or as compared to a known average quantity associated with the characteristic in question. For example, “improved” agronomic traits associated with application of a beneficial microbe, or microbial ensemble, of the disclosure can be demonstrated by comparing the sugar content of plants treated by the microbes taught herein to the sugar content of plants not treated. In the present disclosure, “improved” does not necessarily demand that the data be statistically significant (i.e. p<0.05); rather, any quantifiable difference demonstrating that one value (e.g. the average treatment value) is different from another (e.g. the average control value) can rise to the level of “improved.”

As used herein, “inhibiting and suppressing” and like terms should not be construed to require complete inhibition or suppression, although this may be desired in some embodiments.

The term “marker” or “unique marker” as used herein is an indicator of unique microorganism type, microorganism strain or activity of a microorganism strain. A marker can be measured in biological samples and includes without limitation, a nucleic acid-based marker such as a ribosomal RNA gene, a peptide- or protein-based marker, and/or a metabolite or other small molecule marker.

The term “metabolite” as used herein is an intermediate or product of metabolism. A metabolite in one embodiment is a small molecule. Metabolites have various functions, including in fuel, structural, signaling, stimulatory and inhibitory effects on enzymes, as a cofactor to an enzyme, in defense, and in interactions with other organisms (such as pigments, odorants and pheromones). A primary metabolite is directly involved in normal growth, development and reproduction. A secondary metabolite is not directly involved in these processes but usually has an important ecological function. Examples of metabolites include but are not limited to antibiotics and pigments such as resins and terpenes, etc. Some antibiotics use primary metabolites as precursors, such as actinomycin which is created from the primary metabolite, tryptophan. Metabolites, as used herein, include small, hydrophilic carbohydrates; large, hydrophobic lipids and complex natural compounds.

As used herein, the term “trait” refers to a characteristic or phenotype. For example, in the context of some embodiments of the present disclosure; efficiency of feed utilization, particularly with corn-intensive diets; amount of feces produced; susceptibility to gut pathogens; and a decrease in mortality rates; among others. Desirable traits may also include other characteristics, including but not limited to: an increase in weight; an increase in average daily weight gain; an increase of musculature; an increase of fatty acid concentration in the gastrointestinal tract; an improved efficiency in feed utilization and digestibility; an increase in polysaccharide and lignin degradation: an increase in fat, starch, and/or protein digestion; an increase in fatty acid concentration in the rumen; PH balance in the rumen, an increase in vitamin availability; an increase in mineral availability; an increase in amino acid availability; a reduction in methane and/or nitrous oxide emissions; a reduction in manure production; an improved dry matter intake; an improved efficiency of nitrogen utilization: an improved efficiency of phosphorous utilization: an increased resistance to colonization of pathogenic microbes that colonize cattle; reduced mortality; increased production of antimicrobials; increased clearance of pathogenic microbes; increased resistance to colonization of pathogenic microbes that colonize cattle; increased resistance to colonization of pathogenic microbes that infect humans; reduced incidence of acidosis or bloat; increased meat marbling, increased or decreased red coloring of meat, increased or decreased texture/coarseness of meat; increased amount of USDA Prime, USDA Choice, and USDA Select quality meat per animal, increased in the number of animals producing USDA Prime, USDA Choice, and USDA Select quality meat; increase or reduced concentration or presence of volatile compounds in the meat; reduced prevalence of acidosis or bloat; reduced body temperature; and any combination thereof; wherein said increase or reduction is determined by comparing against an animal not having been administered said composition.

A trait may be inherited in a dominant or recessive manner, or in a partial or incomplete-dominant manner. A trait may be monogenic (i.e. determined by a single locus) or polygenic (i.e., determined by more than one locus) or may also result from the interaction of one or more genes with the environment.

In the context of this disclosure, traits may also result from the interaction of one or more plant genes and one or more microorganism genes.

In the present disclosure, “nucleic acid” refers to a deoxyribonucleotide or ribonucleotide polymer in either single- or double-stranded form, and unless otherwise limited, encompasses known analogues (e.g., peptide nucleic acids) having the essential nature of natural nucleotides in that they hybridize to single-stranded nucleic acids in a manner similar to naturally occurring nucleotides.

The terms “polypeptide,” “peptide,” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers. Polypeptides of the present disclosure can be produced either from a nucleic acid disclosed herein, or by the use of standard molecular biology techniques. For example, a truncated protein of the present disclosure can be produced by expression of a recombinant nucleic acid of the embodiments in an appropriate host cell, or alternatively by a combination of ex vivo procedures, such as protease digestion and purification.

The term “encode” is used herein to mean that the nucleic acid comprises the required information, specified by the use of codons to direct translation of the nucleotide sequence into a specified protein. A nucleic acid encoding a protein can comprise non-translated sequences (e.g., introns) within translated regions of the nucleic acid or can lack such intervening non-translated sequences (e.g., as in cDNA).

Aspects of the disclosure encompass isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material, or culture medium when produced by recombinant techniques (e.g. PCR amplification), or substantially free of chemical precursors or other chemicals when chemically synthesized. Optimally, an “isolated” polynucleotide is free of sequences (for example, protein encoding sequences) that naturally flank the polynucleotide (i.e., sequences located at the 5′ and 3′ ends of the polynucleotide) in the genomic DNA of the organism from which the polynucleotide is derived. For example, in some aspects of the disclosure, the isolated polynucleotide can contain less than about 5 kb, about 4 kb, about 3 kb, about 2 kb, about 1 kb, about 0.5 kb, or about 0.1 kb of nucleotide sequence that naturally flank the polynucleotide in genomic DNA of the cell from which the polynucleotide is derived. A protein that is substantially free of cellular material includes preparations of protein having less than about 30%, about 20%, about 10%, about 5%, or about 1% (by dry weight) of contaminating protein. When the protein of the aspects, or a biologically active portion thereof, is recombinantly produced, optimally culture medium represents less than about 30%, about 20%, about 10%, about 5%, or about 1% (by dry weight) of chemical precursors or non-protein-of-interest chemicals.

The polynucleotides described herewith can be used to isolate corresponding sequences from other organisms, particularly other plants. In this manner, methods such as PCR or hybridization can be used to identify such sequences based on their sequence homology to the sequences set forth herein. Sequences isolated based on their sequence identity to the entire sequences set forth herein or to variants and fragments thereof are encompassed by the present disclosure. Such sequences include sequences that are orthologs of the disclosed sequences. The term “orthologs” refers to genes derived from a common ancestral gene and which are found in different species as a result of speciation. Genes found in different species are considered orthologs when their nucleotide sequences and/or their encoded protein sequences share at least about 60%, about 70%, about 75%, about 80%, about 85%, about 90%, about 91%, about 92%, about 93%, about 94%, about 95%, about 96%, about 97%, about 98%, about 99%, or greater sequence identity. Functions of orthologs are often highly conserved among species. Thus, isolated polynucleotides that encode for a protein that confers or enhances fungal plant pathogen resistance and that hybridize to the sequences disclosed herein, or to variants or fragments thereof, are encompassed by the present disclosure.

The terms “increase,” “increasing.” “enhance,” “enhancing” and the like are used herein to mean any boost or gain or rise in the amount of a composition (e.g., sugar content). Further, the terms “induce” or “increase” as used herein can mean higher concentration of an amount of a composition (e.g., sugar content), such that the level is increased 5% or more, 10% or more, 50% or more or 100% relative to a control subject or target.

The term “expression” as used herein in refers to the biosynthesis or process by which a polynucleotide, for example, is produced, including the transcription and/or translation of a gene product. For example, a polynucleotide of the present disclosure can be transcribed from a DNA template (such as into an mRNA or other RNA transcript) and/or the process by which a transcribed mRNA is subsequently translated into a polypeptide or protein. The term “gene product” can refer to for example, transcripts and encoded polypeptides. Inhibition of (or increase in) expression or function of a gene product (i.e., a gene product of interest) can be in the context of a comparison between any two plants, for example, expression or function of a gene product in a genetically altered plant versus the expression or function of that gene product in a corresponding, but susceptible wild-type plant or other susceptible plant. The expression level of a gene product in a wild-type plant can be absent.

Alternatively, inhibition of (or increase in) expression or function of the target gene product can be in the context of a comparison between plant cells, organelles, organs, tissues, or plant parts within the same plant or between plants, and includes comparisons between developmental or temporal stages within the same plant or between plants. Any method or composition that down-regulates expression of a target gene product, either at the level of transcription or translation, or down-regulates functional activity of the target gene product can be used to achieve inhibition of expression or function of the target gene product. Similarly, any method or composition that induces or up-regulates expression of a target gene product, either at the level of transcription or translation, or increases or activates or up-regulates functional activity of the target gene product can be used to achieve increased expression or function of the target gene or protein. Methods for inhibiting or enhancing gene expression are well known in the art.

As used herein “shelf-stable” refers to a functional attribute and new utility acquired by the microbes formulated according to the disclosure, which enable said microbes to exist in a useful/active state outside of their natural environment in the rumen (i.e. a markedly different characteristic). Thus, shelf-stable is a functional attribute created by the formulations/compositions of the disclosure and denoting that the microbe formulated into a shelf-stable composition can exist outside the rumen and under ambient conditions for a period of time that can be determined depending upon the particular formulation utilized, but in general means that the microbes can be formulated to exist in a composition that is stable under ambient conditions for at least a few days and generally at least one week. Accordingly, a “shelf-stable ruminant supplement” is a composition comprising one or more microbes of the disclosure, said microbes formulated in a composition, such that the composition is stable under ambient conditions for at least one week, meaning that the microbes comprised in the composition (e.g. whole cell, spore, or lysed cell) are able to impart one or more beneficial phenotypic properties to a ruminant when administered (e.g. increased milk yield, improved milk compositional characteristics, improved rumen health, and/or modulation of the rumen microbiome).

“Percentage of sequence identity”, as used herein, is determined by comparing two optimally locally aligned sequences over a comparison window defined by the length of the local alignment between the two sequences. The amino acid sequence in the comparison window may comprise additions or deletions (e.g., gaps or overhangs) as compared to the reference sequence (which does not comprise additions or deletions) for optimal alignment of the two sequences. Local alignment between two sequences only includes segments of each sequence that are deemed to be sufficiently similar according to a criterion that depends on the algorithm used to perform the alignment (e. g. BLAST). The percentage of sequence identity is calculated by determining the number of positions at which the identical nucleic acid base or amino acid residue occurs in both sequences to yield the number of matched positions, dividing the number of matched positions by the total number of positions in the window of comparison and multiplying the result by 100. Optimal alignment of sequences for comparison may be conducted by the local homology algorithm of Smith and Waterman (Add. APL. Math. 2:482, 1981), by the global homology alignment algorithm of Needleman and Wunsch (J Mol. Biol. 48:443, 1970), by the search for similarity method of Pearson and Lipman (Proc. Natl. Acad. Sci. USA 85:2444, 1988), by heuristic implementations of these algorithms (NCBI BLAST. WU-BLAST. BLAT. SIM, BLASTZ), or by inspection. Given that two sequences have been identified for comparison. GAP and BESTFIT are preferably employed to determine their optimal alignment. Typically, the default values of 5.00 for gap weight and 0.30 for gap weight length are used. The term “substantial sequence identity” between polynucleotide or polypeptide sequences refers to polynucleotide or polypeptide comprising a sequence that has at least 50% sequence identity, preferably at least 70%, preferably at least 80%>, preferably at least 85%, preferably at least 90%>, preferably at least 95%, and preferably at least 96%>, 97%, 98% or 99% sequence identity compared to a reference sequence using the programs. In addition, pairwise sequence homology or sequence similarity, as used, refers to the percentage of residues that are similar between two sequences aligned. Families of amino acid residues having similar side chains have been well defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Query nucleic acid and amino acid sequences can be searched against subject nucleic acid or amino acid sequences residing in public or proprietary databases. Such searches can be done using the National Center for Biotechnology Information Basic Local Alignment Search Tool (NCBI BLAST v 2.18) program. The NCBI BLAST program is available on the internet from the National Center for Biotechnology Information (blast.ncbi.nlm.nih.gov/Blast.cgi). Typically the following parameters for NCBI BLAST can be used: Filter options set to “default”, the Comparison Matrix set to “BLOSUM62”, the Gap Costs set to “Existence: 11, Extension: 1”, the Word Size set to 3, the Expect (E threshold) set to 1e-3, and the minimum length of the local alignment set to 50% of the query sequence length. Sequence identity and similarity may also be determined using GenomeQuest™ software (Gene-IT, Worcester Mass. USA).

A “control plant”, as used herein, provides a reference point for measuring changes in phenotype of the subject plant, and may be any suitable plant cell, seed, plant component, plant tissue, plant organ or whole plant which has not been exposed to a particular treatment such as, for example, an inoculant or combination of inoculants and/or other chemicals.

“Inoculant” as used herein refers to any culture or preparation that comprises at least one microorganism. In some aspects, an inoculant (sometimes as microbial inoculant, or soil inoculant) is an agricultural amendment that uses beneficial microbes (including, but not limited to endophytes) to promote plant health, growth and/or yield, animal health, growth or improvement of one or more traits. Many of the microbes suitable for use in an inoculant form symbiotic relationships with the target crops where both parties benefit (mutualism).

A “plant element” as used herein refer to either a whole plant or a plant component, including but not limited to plant tissues, parts, and cell types. A plant element can be one of the following: whole plant, seedling, meristematic tissue, ground tissue, vascular tissue, dermal tissue, seed, leaf, root, shoot, stem, flower, fruit, stolon, bulb, tuber, corm, kelkis, shoot, bud. As used herein, a “plant element” is synonymous to a “portion” of a plant, and refers to any part of the plant, and can include distinct tissues and/or organs, and may be used interchangeably with the term “tissue” throughout.

As used herein, the phrase “plant reproductive element” means any part of a plant that is able to initiate other plants via either sexual or asexual reproduction of that plant, for example but not limited to: seed, seedling, root, shoot, stolon, bulb, tuber, corm, keikis, or bud.

### Compositions

Described herein are microbial inoculant compositions comprising aquatic microbial species for application to terrestrial plants. In some aspects, the inoculant mixture also comprises a species that produces and/or maintains a microenvironment in the plant that is suitable for other microbes in the inoculant mixture to thrive.

Disclosed herein are compositions comprising a plant seed and one or more of the microbes listed in Table 1, Table 2, Table 3 or Table 4.

Disclosed herein are compositions comprising a plant seed and two or more bacterial strains. In some aspects, a first bacterial strain comprises Clostridium spp. In some aspects, the 16S sequence of Clostridium spp. comprises any one of the Clostridium spp. listed in Table 1 or Table 2. In some aspects, a second bacterial strain comprises an aquatic Pseudomonas spp. with a 16S nucleic acid sequence that is at least about 97% identical to any one of the Pseudomonas spp. listed in Table 1 or Table 2.

Disclosed herein are compositions comprising one or more of the microbes listed in Table 1, Table 2, Table 3 or Table 4. In some aspects, the compositions disclosed herein can further comprise one or more of the microbes listed in Table 1, Table 2, Table 3, or Table 4. In some aspects, the compositions disclosed herein can further comprise at least one different microbial strain. In some aspects, the 16S sequence of the one different microbial strain can comprise a 16S sequence that is at least about 97% identical to one or more of the 16S sequences listed in Table 1, Table 2, Table 3, or Table 4.

In some aspects, the compositions disclosed herein can further comprise an agriculturally effective amount of a compound or composition selected from the group consisting of a nutrient, a fertilizer, an acaricide, a bactericide, a fungicide, an insecticide, a microbicide, a nematicide, and a pesticide.

In some aspects, the compositions disclosed herein can further comprise a carrier. In some aspects, the carrier can be peat, turf, talc, lignite, kaolinite, pyrophyllite, zeolite, montmorillonite, alginate, press mud, sawdust, perlite, mica, silicas, quartz powder, calcium bentonite, vermiculite or mixtures thereof.

In some aspects, the compositions disclosed herein can be prepared as a formulation selected from the group consisting of an emulsion, a colloid, a dust, a granule, a pellet, a powder, a spray, and a solution.

In some aspects, compositions disclosed herein can be mixed with animal feed. In some aspects, the animal feed can be present in various forms such as pellets, capsules, granulated, powdered, mash, liquid, semi-liquid, or mixed rations(s).

In some aspects, the plant seed can be a transgenic plant seed.

Disclosed herein are plant seeds. In some aspects, the plants seeds can have a coating comprising any of the compositions disclosed herein. In some aspects, the plant seeds can have a coating comprising two or more bacterial strains, wherein a first bacterial strain comprises Clostridium spp., and wherein the 16S sequence of Clostridium spp. comprises any one of the Clostridium spp. listed in Table 1 or Table 2 and a second bacterial strain comprising an aquatic Pseudomonas spp. with a 16S nucleic acid sequence that is at least about 97% identical to any one of the Pseudomonas spp. listed in Table 1 or Table 2. In some aspects, the plant seeds can have a coating comprising two or more bacterial strains, wherein a first bacterial strain comprises Clostridium spp., and wherein the 16S sequence of Clostridium spp. comprises any one of the Clostridium spp. listed in Table 1 or Table 2, a second bacterial strain comprising an aquatic Pseudomonas spp. with a 16S nucleic acid sequence that is at least about 97% identical to any one of the Pseudomonas spp. listed in Table 1 or Table 2 and one or more of the microbes listed in Table 1, Table 2 or Table 3. In some aspects, the plant seeds can have a coating comprising two or more bacterial strains, wherein a first bacterial strain comprises Clostridium spp., and wherein the 16S sequence of Clostridium spp. comprises any one of the Clostridium spp. listed in Table 1 or Table 2, a second bacterial strain comprising an aquatic Pseudomonas spp. with a 16S nucleic acid sequence that is at least about 97% identical to any one of the Pseudomonas spp. listed in Table 1 or Table 2 and one or more of the microbes listed in Table 1, Table 2 or Table 3. In some aspects, the plant seeds can a coating further comprise a composition that has at least one different microbial strain, wherein the 16S sequence of the one different microbial strain comprises a 16S sequence that is at least about 97% identical to one or more of the 16S sequences listed in Table 1, Table 2 or Table 3.

The primary structure of major rRNA subunit 16S comprise a particular combination of conserved, variable, and hypervariable regions that evolve at different rates and enable the resolution of both very ancient lineages such as domains, and more modern lineages such as genera. The secondary structure of the 16S subunit include approximately 50 helices which result in base pairing of about 67% of the residues. These highly conserved secondary structural features are of great functional importance and can be used to ensure positional homology in multiple sequence alignments and phylogenetic analysis. Over the previous few decades, the 16S rRNA gene has become the most sequenced taxonomic marker and is the cornerstone for the current systematic classification of bacteria and archaea (Yarza et al. 2014. Nature Rev. Micro. 12:635-45).

A sequence identity of 94.5% or lower for two 16S rRNA genes is strong evidence for distinct genera, 86.5% or lower is strong evidence for distinct families, 82% or lower is strong evidence for distinct orders, 78.5% is strong evidence for distinct classes, and 75% or lower is strong evidence for distinct phyla. The comparative analysis of 16S rRNA gene sequences enables the establishment of taxonomic thresholds that are useful not only for the classification of cultured microorganisms but also for the classification of the many environmental sequences. Yarza et al. 2014. Nature Rev. Micro. 12:635-45).

A loss of biodiversity within a soil matrix can lead to yield depression of agricultural crops. Microbial inoculants can increase solubilization, uptake, and/or assimilation of nutrients such as, for example, carbon, nitrogen, potassium, phosphorus, selenium, cobalt, zinc, and copper. Microbial inoculants also can reduce plant pathogen damage to crops by stimulating plant production of a stable and continuous source of plant hormones that enhance growth. While microorganisms capable of promoting plant growth and plant production can occur naturally in soil, the mere presence of the microbes does not guarantee the successful integration of the microbes.

In some aspects, the microbial inoculant composition can function endophytically within at least one plant to maintain an available electron state that is available for use within the plant's metabolic process. That is, the microbial inoculant composition can act as an ionic catalyst to either accept or remove an electron to make the electron available to or remove the electron from the plant. This process can occur, in the absence of such a microbial inoculant composition, when a plant switches from photosynthesis during the day to respiration at night and vice versa. The microbial inoculant composition, when applied to the plant, supports the plant by making nutrients chemically available so the plant can produce hormones at a sufficient level to promote growth.

The microbial inoculant composition can inoculate the plant by being in close proximity and/or direct physical contact with the plant. As an example, a droplet of water including the microbial inoculant composition can be deposited on the plant, and thereby not deposited in the soil and not absorbed by the roots.

Described herein are microbial inoculant compositions isolated from an aquatic environment for application to terrestrial plants. In some aspects, the inoculant mixture also comprises a species that produces and/or maintains a microenvironment in the plant that is suitable for other microbes in the inoculant mixture to thrive.

Generally, the microbial inoculant composition includes a Pseudomonas spp. and a Clostridium spp., such as, for example, P. fluorescens and C. saccharobutylicum.

In some aspects, the microbial inoculant composition further comprises one or more of Agrobacterium tume aciens (TPD7005), Bacillus megaterium (TPD7007), Bacillus megaterium (TPD 7008), Agrobacterium rhizogenes (1713117 009), Microbacterium testaceum (TPD7010), Bacillus megaterium (TPD7011), Microbacterium spp. (TPD7012), Pedobacier kribbensis (TPD70013), Janthinobacterium lividum (TPD7014), Bacillus racemilacticus (TPD7015), Bacillus megaterium (TPD 7018), Delftia spp. (TPD3002), Chryseobacterium spp. (TPD3003), Bacillus licheniformis, Brevundimonas kwangchunensis (TPD3004), Fictibacillus barbaricus/Bacillus barbaricus (TPD3005), Prosthecobacter spp. (TPD3006), Lactobacillus plantarum (TPD3007), Sphingobacterium multivorum, Sphingomonas spp. (TPD3009), Sphingosinicella microcystinivorans (TPD3010), Pseudomonas chlororaphis, Pseudomonas mandelii, Pseudomonas umsongensis, Clostridium saccharobutylicum (TPD3014), Arthrobacter ramosus (TPD3015), Streptomyces yogyakartensis (TPD3016), Arthrobacter spp. (TPD3017), Xanthomonas spp., Chryseobacterium indologenes (TPD3019), or Lactobacillus plantarum.

Table 1 shows 16S RNA analysis and/or whole genome shotgun sequencing project data for exemplary members of the microbial inoculant composition.

TABLE 1
Microbes

| Species | Designation | GenBank Accession No. |
|---|---|---|
| Pseudomonas veronii | TPD3012 | MH190219.1 |
| Pseudomonas mandelii | TPD3013 | MH221124.1 |
| Pseudomonas mandelii | | CP005960.1 |
| Pseudomonas moraviensis | TPD3001 | MH190053.1 |
| Pseudomonas protegens | TPD3011 | MH221127.1 |
| Pantoea agglomerans | TPD7001 | MH190052.1 |
| Pantoea agglomerans | | CP016889.1 |
| Clostridium saccharobutylicum | TPD3014 | MH189851.1 |
| Clostridium saccharobutylicum | TPD7003 | MH192394.1 |
| Erwinia aphidicola | TPD7004 | MH190220.1 |
| Serratia liquefaciens | TPD7002 | MH190215.1 |
| Pedobacter kribbensis | TPD70013 | MH221086.1 |
| Janthinobacterium lividum | TPD7014 | MH221099.1 |
| Bacillus racemilacticus | TPD7015 | MH221098.1 |
| Sphingomonas spp. | TPD3009 | QDFK00000000.1 |
| Sphingomonas sp. | | CP015521.1 |
| Agrobacterium tumefaciens | TPD7005 | QDFL00000000.1 |
| Agrobacterium tumefaciens | | AE007869.2 |
| Bacillus megaterium | TPD7018 | QDFM00000000.1 |
| Sphingomonas spp. | TPD3009 | QDFN 0000000.1 |
| Bacillus megaterium | TPD7007 | QDFO00000000.1 |
| Bacillus megaterium | TPD7008 | QDFP00000000.1 |
| Bacillus megaterium | | CP001983.1 |
| Arthrobacter spp. | TPD3018 | QDFQ00000000.1 |
| Arthrobacter sp. | | CP022436.1 |
| Agrobacterium rhizogenes | TPD7009 | QDFR00000000.1 |
| Agrobacterium rhizogenes | | CP019701.1 |
| Sphingomonas melonis | TPD3008 | QDFS00000000.1 |
| Sphingomonas melonis | | CP023705.1 |
| Microbacterium testaceum | TPD7010 | QDFT00000000.1 |
| Microbacterium testaceum | | AP012052.1 |
| Bacillus megaterium | TPD7011 | QDFU00000000.1 |
| Microbacterium spp. | TPD7012 | QDFV00000000.1 |
| Microbacterium sp. | | AP017975.1 |

Table 2 shows bacterial strains useful in the compositions and methods disclosed herein.

TABLE 2
Microbes.

| Species | GenBank Accession No. |
|---|---|
| arthrobacter ramosus | CP022436.1 |
| arthrobacter sp. | CP022436.1 |
| brevundimonas kwangchunensis | |
| chryseobacterium sp. | |
| clostridium spp. | |
| clostridium uliginosum | |
| delftia spp. | |
| fictibacillus bacillus barbaricus | |
| lactobacillus plantarum | |
| prosthecobacter | |
| pseudomonas chlororaphis | |
| pseudomonas mandelii | CP005960.1 |
| pseudomonas spp. | LT707063.1 |
| pseudomonas umsongensis | |
| sphingobacterium multivorum | |
| sphingomonas sp. | CP015521.1 |
| sphingosinicella microcystinivorans | |
| streptomyces yogyakartansis | |

Table 3 shows bacterial strains useful in the compositions and methods disclosed herein.

TABLE 3
Microbes.

| Species | GenBank Accession No. |
|---|---|
| acetivibrio cellulolyticus | |
| acetobacteraceae | |
| acidimicrobiaceae | |
| acidimicrobiales | |
| acidimicrobium spp. | |
| acidiphilium | |
| aciditerrimonas | |
| aciditerrimonas sp. | |
| aciditerrimonas spp. | |
| acidobacteria | |
| acidobacteriaceae | |
| acidobacteriales | |
| acidobacteriia | |
| acidobacterium | |
| acidobacterium spp. | |
| acidovorax | |
| acidovorax citrulli | |
| acinetobacter lwoffii | |
| actinoallomurus iriomotensis | |
| actinobacteria | |
| actinomadura | |
| actinomadura sp. | |
| actinomyces | |
| actinomycetales | |
| actinopolymorpha | |
| actinopolymorpha pittospori | |
| actinotalea fermentans | |
| adhaeribacter spp. | |
| adhaeribacter terreus | |
| aeromicrobium fastidiosum | |
| aeromicrobium spp. | |
| afipia sp. | |
| afipia spp. | |
| agromyces subbeticus | |
| agromyces ulmi | |
| alcaligenaceae | |
| algoriphagus sp. | |
| alphaproteobacteria | |
| altererythrobacter alterierythrobacter sp. | |
| altererythrobacter sp. | |
| altererythrobacter spp. | |
| alteromonadaceae | |
| amaricoccus sp. | |
| aminobacter sp. | |
| amorphus | |
| amycolatopsis | |
| amycolatopsis iriomotensis | |
| amycolatopsis spp. | |
| amycolatopsis vancoresmycina | |
| anaerolineales | |
| anaeromyxobacter | |
| anaeromyxobacter spp. | |
| anaeromyxobacteraceae | |
| ancylobacter | |
| ancylobacter spp. | |
| angustibacter peucedani | |
| aquabacterium spp. | |
| aquicella | |
| arenimonas oryziterrae | |
| armatimonadetes | |
| arsenicicoccus | |
| arsenicicoccus dermatophilus sp. | |
| arthrobacter | |
| arthrobacter pascens | |
| arthrobacter tumbae | |
| asanoa ishikariensis | |
| azohydromonas australica | |
| azonexus sp. | |
| azospira | |
| azospira oryzae | |
| azospira spp. | |
| azospirillum lipoferum | |
| azotobacter chroococcum | |
| bacillaceae | |
| bacillales | |
| bacilli | |
| bacillus | |
| bacillus acidiceler | |
| bacillus senegalensis | |
| bacillus sp. | |
| bacillus spp. | |
| bacteroidales | |
| bauldia | |
| bauldia consociata | |
| bdellovibrionaceae | |
| beijerinckia spp. | |
| blastococcus sp. | |
| blastococcus spp. | |
| blastomonas | |
| blastomonas spp. | |
| bordetella hinzii | |
| bosea sp. | |
| bradyrhizobiaceae | |
| bradyrhizobium elkanii | |
| bradyrhizobium sp. | |
| bradyrhizobium spp. | |
| bradyrhizobium yuanmingense | |
| brevundimonas | |
| brevundimonas lenta | |
| brucellaceae | |
| bryobacter | |
| burkholderia | |
| burkholderia sp. | |
| burkholderia spp. | |
| burkholderiaceae | |
| burkholderiales | |
| buttiauxella izardii | |
| byssovorax | |
| caldilinea | |
| caldilinea spp. | |
| caldilineaceae | |
| caldilineales | |
| candidatus brocadiaceae | |
| candidatus koribacter | |
| candidatus nitrosoarchaeum | |
| candidatus nitrosoarchaeum limnia | |
| candidatus phytoplasma phytoplasma sp. | |
| ryl_gd | |
| candidatus saccharibacteria | |
| candidatus solibacter | |
| candidatus solibacter uncultured solibacter | |
| sp. | |
| candidatus solibacter usitatus | |
| carnobacterium spp. | |
| catellatospora citrea | |
| catellatospora sp. | |
| catellatospora spp. | |
| catenuloplanes spp. | |
| caulobacter sp. | |
| caulobacter tundrae | |
| caulobacteraceae | |
| caulobacterales | |
| cellulomonas terrae | |
| cellvibrio vulgaris | |
| chelatococcus asaccharovorans | |
| chelatococcus spp. | |
| chitinophagaceae | |
| chloroflexaceae | |
| chloroflexales | |
| chloroflexi | |
| chloroflexia | |
| chloroflexus | |
| chloroflexus spp. | |
| chromobacteriaceae | |
| chryseobacterium | |
| chryseobacterium indologenes | |
| chthoniobacter flavus | |
| citrobacter spp. | |
| clavibacter michiganensis | |
| clostridia | |
| clostridiaceae | |
| clostridiales | |
| clostridium | |
| clostridium bowmanii | |
| clostridium gasigenes | |
| clostridium spp. | |
| clostridium vincentii | |
| comamonadaceae | |
| comamonas | |
| comamonas koreensis | |
| conexibacter | |
| conexibacter spp. | |
| conexibacter woesei | |
| conexibacteraceae | |
| coxiellaceae | |
| crenotrichaceae | |
| cryobacterium mesophilum | |
| cryobacterium sp. | |
| cryomorphaceae | |
| cupriavidus | |
| cupriavidus campinensis | |
| cyanobacteria | |
| cystobacter sp. | |
| cystobacter spp. | |
| cystobacteraceae | |
| cytophaga spp. | |
| cytophagaceae | |
| cytophagales | |
| dehalococcoidales | |
| dehalococcoides | |
| dehalococcoidia | |
| dehalogenimonas spp. | |
| denitratisoma | |
| denitratisoma spp. | |
| derxia | |
| derxia spp. | |
| desulfatiglans | |
| desulfobacteraceae | |
| desulfoglaeba spp. | |
| desulfosporosinus meridiei | |
| desulfuromonadaceae | |
| desulfuromonadales | |
| desulfuromonas | |
| devosia insulae | |
| devosia sp. | |
| devosia spp. | |
| dickeya zeae | |
| dyadobacter sp. | |
| elusimicrobia | |
| elusimicrobiaceae | |
| elusimicrobiales | |
| endomicrobia | |
| enhygromyxa salina | |
| epilithonimonas sp. | |
| erwinia persicina | |
| exiguobacterium undae | |
| ferrimicrobium | |
| ferrimicrobium spp. | |
| fictibacillus | |
| flavisolibacter spp. | |
| flavobacteriaceae | |
| flavobacteriales | |
| flavobacterium | |
| flavobacterium arsenatis | |
| flavobacterium columnare | |
| flavobacterium hauense | |
| flavobacterium johnsoniae | |
| flavobacterium sp. | |
| flavobacterium spp. | |
| flavobacterium terrigena | |
| flexibacter | |
| flexibacter spp. | |
| fodinicola spp. | |
| frankia | |
| frankia spp. | |
| frankiaceae | |
| frigoribacterium sp. | |
| gaiella | |
| gaiella occulta | |
| gaiella spp. | |
| gaiellaceae | |
| gaiellales | |
| gallionella | |
| gallionellaceae | |
| gammaproteobacteria | |
| gemmatimonadaceae | |
| gemmatimonadales | |
| gemmatimonas | |
| gemmatimonas sp. | |
| gemmatimonas spp. | |
| geobacillus sp. | |
| geobacter | |
| geobacter sp. | |
| geobacter spp. | |
| geobacteraceae | |
| halomonas muralis | |
| herbaspirillum huttiense | |
| herbaspirillum sp. | |
| herbaspirillum spp. | |
| holophaga | |
| holophaga spp. | |
| holophagaceae | |
| humibacillus xanthopallidus | |
| hydrogenophaga palleronii | |
| hydrogenophilaceae | |
| hyphomicrobiaceae | |
| hyphomicrobium | |
| hyphomicrobium sp. | |
| hyphomonas | |
| iamia | |
| iamia sp. | |
| iamia spp. | |
| iamiaceae | |
| ideonella sp. | |
| ignavibacteriaceae | |
| ignavibacteriales | |
| ignavibacterium | |
| ignavibacterium spp. | |
| ilumatobacter | |
| ilumatobacter spp. | |
| intrasporangium oryzae | |
| jiangella | |
| kaistia | |
| kallotenuales | |
| kineococcus sp. | |
| kineosporia mikuniensis | |
| kofleria | |
| kofleria spp. | |
| kofleriaceae | |
| kribbella karoonensis | |
| kribbella sp. | |
| kribbella swartbergensis | |
| labedella sp. | |
| labilithrix luteola | |
| labilitrichaceae | |
| lactobacillus | |
| lactococcus garvieae | |
| lapillicoccus jejuensis | |
| legionellaceae | |
| leifsonia kribbensis | |
| leifsonia spp. | |
| lentzea albida | |
| leptothrix sp. | |
| leptothrix spp. | |
| leucobacter tardus | |
| lysinibacillus sphaericus | |
| lysobacter sp. | |
| lysobacter spp. | |
| marinimicrobium | |
| marinobacter | |
| massilia | |
| massilia sp. | |
| massilia timonae | |
| melioribacter | |
| melioribacter spp. | |
| melioribacteraceae | |
| mesorhizobium loti | |
| mesorhizobium plurifarium | |
| mesorhizobium sp. | |
| mesorhizobium spp. | |
| methylibium | |
| methylobacillus flagellatus | |
| methylobacillus spp. | |
| methylobacter spp. | |
| methylobacteriaceae | |
| methylobacterium adhaesivum | |
| methylobacterium spp. | |
| methylocella spp. | |
| methylococcaceae | |
| methylococcales | |
| methyloversatilis | |
| methyloversatilis spp. | |
| microbacteriaceae | |
| microbacterium kitamiense | |
| microbacterium sp. | AP017975.1 |
| microcella alkaliphila | |
| micrococcaceae | |
| microlunatus spp. | |
| micromonospora rhodorangea | |
| micromonosporaceae | |
| microvirga aerilata | |
| microvirga subterranea | |
| moorella spp. | |
| mycobacterium sacrum | |
| mycobacterium salmoniphilum | |
| mycobacterium septicum | |
| mycobacterium spp. | |
| nakamurella sp. | |
| nannocystaceae | |
| nannocystis | |
| nannocystis exedens | |
| neorhizobium rhizobium huautlense | |
| niastella spp. | |
| nitrosomonadaceae | |
| nitrosomonas spp. | |
| nitrosomonas ureae | |
| nitrosopumilaceae | |
| nitrosospira | |
| nitrosospira spp. | |
| nitrosovibrio tenuis | |
| nitrospira enrichment | |
| nitrospira sp. | |
| nitrospira spp. | |
| nitrospiraceae | |
| nitrospirales | |
| nocardia anaemiae | |
| nocardia pneumoniae | |
| nocardioidaceae | |
| nocardioides iriomotensis | |
| nocardioides islandensis | |
| nocardioides maritimus | |
| nocardioides perillae | |
| nocardioides sp. | |
| nocardioides spp. | |
| nordella | |
| nordella spp. | |
| novosphingobium sp. | |
| novosphingobium spp. | |
| ochrobactrum haematophilum | |
| ohtaekwangia spp. | |
| olivibacter soli | |
| opitutaceae | |
| oryzihumus spp. | |
| oxalobacteraceae | |
| paenibacillaceae | |
| paenibacillus | |
| paenibacillus sp. | |
| pantoea agglomerans | CP016889.1 |
| paracoccus spp. | |
| paracraurococcus sp. | |
| parastreptomyces | |
| pasteuriaceae | |
| pedobacter kribbensis | |
| pedobacter kwangyangensis | |
| pedobacter sp. | |
| pedobacter spp. | |
| pedobacter tournemirensis | |
| pedosphaera | |
| pedosphaera spp. | |
| pelobacter | |
| pelobacter spp. | |
| peredibacter spp. | |
| phaselicystidaceae | |
| phenylobacterium | |
| phenylobacterium sp. | |
| phenylobacterium spp. | |
| phycicoccus sp. | |
| phycisphaerae | |
| phycisphaerales | |
| phyllobacterium | |
| phyllobacterium spp. | |
| phyllobacterium trifolii | |
| pigmentiphaga sp. | |
| pirellula spp. | |
| planctomycetaceae | |
| planctomycetales | |
| planctomycetia | |
| planococcus spp. | |
| plesiocystis spp. | |
| polaromonas spp. | |
| procabacteriales | |
| promicromonospora sp. | |
| promicromonospora sukumoe | |
| prosthecobacter spp. | |
| prosthecomicrobium spp. | |
| pseudoalteromonas | |
| pseudoclavibacter helvolus | |
| pseudolabrys | |
| pseudolabrys spp. | |
| pseudolabrys taiwanensis | |
| pseudomonadaceae | |
| pseudomonadales | |
| pseudomonas | |
| pseudomonas flavescens | |
| pseudomonas fluorescens | |
| pseudonocardia | |
| pseudonocardia carboxydivorans | |
| pseudonocardia sp. | |
| pseudonocardia spp. | |
| pseudonocardia zijingensis | |
| pseudorhodoferax sp. | |
| pseudoxanthobacter | |
| pseudoxanthomonas spp. | |
| ralstonia spp. | |
| ramlibacter sp. | |
| ramlibacter spp. | |
| reyranella massiliensis | |
| reyranella sp. | |
| rheinheimera sp. | |
| rhizobiaceae | |
| rhizobiales | |
| rhizobium | |
| rhizobium etli | |
| rhizobium sp. | |
| rhizobium spp. | |
| rhizomicrobium spp. | |
| rhodobacter spp. | |
| rhodobiaceae | |
| rhodococcus kroppenstedtii | |
| rhodococcus spp. | |
| rhodococcus wratislaviensis | |
| rhodocyclaceae | |
| rhodocyclales | |
| rhodomicrobium | |
| rhodomicrobium spp. | |
| rhodoplanes | |
| rhodoplanes sp. | |
| rhodoplanes spp. | |
| rhodopseudomonas spp. | |
| rhodospirillaceae | |
| rhodospirillales | |
| rhodothermus | |
| rickettsiaceae | |
| roseateles | |
| roseateles spp. | |
| rubrivivax | |
| rubrivivax gelatinosus | |
| rubrivivax spp. | |
| rubrobacter | |
| ruminococcaceae | |
| saccharopolyspora | |
| saccharopolyspora gloriosa | |
| saccharopolyspora sp. | |
| sandaracinus | |
| saprospiraceae | |
| serratia proteamaculans | CP000826.1 |
| shimazuella | |
| shinella granuli | |
| sideroxydans lithotrophicus | |
| sideroxydans paludicola | |
| sinobacteraceae | |
| sinorhizobium sp. | |
| solibacteraceae | |
| solirubrobacter | |
| solirubrobacter spp. | |
| sorangium | |
| sorangium cellulosum | |
| sphaerobacteraceae | |
| sphaerobacterales | |
| sphingobacteriaceae | |
| sphingobacteriales | |
| sphingobacterium | |
| sphingobium herbicidovorans | |
| sphingobium xenophagum | |
| sphingomonadaceae | |
| sphingomonadales | |
| sphingomonas | |
| sphingomonas spp. | |
| sphingomonas wittichii | |
| sphingopyxis macrogoltabida | |
| sphingosinicella | |
| sporichthya | |
| sporichthya spp. | |
| sporichthyaceae | |
| stackebrandtia nassauensis | |
| stenotrophomonas maltophilia | |
| steroidobacter | |
| steroidobacter spp. | |
| stigmatella erecta | |
| streptomyces | |
| streptomyces aculeolatus | |
| streptomyces fradiae | |
| streptomyces ghanaensis | |
| streptomyces hebeiensis | |
| streptomyces mashuensis | |
| streptomyces microflavus | |
| streptomyces netropsis | |
| streptomyces sp. | |
| streptomyces spp. | |
| streptomyces variabilis | |
| streptomyces vayuensis | |
| streptomyces viridochromogenes | |
| streptomyces viridodiastaticus | |
| streptomyces xinghaiensis | |
| streptomyces xylophagus | |
| streptomycetaceae | |
| sulfuricella | |
| syntrophaceae | |
| syntrophobacter wolinii | |
| syntrophorhabdaceae | |
| syntrophorhabdus | |
| syntrophus spp. | |
| taibaiella sp. | |
| tepidamorphus | |
| tepidamorphus spp. | |
| terrabacter | |
| terrabacter sp. | |
| terrabacter spp. | |
| terriglobus | |
| terrimonas sp. | |
| terrimonas spp. | |
| tetrasphaera | |
| tetrasphaera elongata | |
| thermomonosporaceae | |
| thiobacillus | |
| thiobacillus denitrificans | |
| thiobacillus spp. | |
| thiobacter spp. | |
| thiomonas | |
| thiorhodovibrio spp. | |
| uncultured candidatus koribacter sp. | |
| variovorax paradoxus | |
| verrucomicrobia subdivision 3 | |
| verrucomicrobiaceae | |
| verrucomicrobiales | |
| woodsholea | |
| woodsholea maritima | |
| xanthomonadaceae | |
| xanthomonadales | |
| xanthomonas | |
| xanthomonas spp. | |
| zoogloea | |
| zooshikella | |

Table 4 shows bacterial strains useful in the compositions and methods disclosed herein.

TABLE 4
Microbes.

| Species | GenBank Accession No. |
|---|---|
| Agrobacterium tumefaciens | AE007869.2 |
| Arthrobacter sp. | CP022436.1 |
| Agrobacterium rhizogenes | CP019701.1 |
| Bacillus megaterium | CP001983.1 |
| Bacillus megaterium | CP018874.1 |
| Erwinia sp. | CP002124.1 |
| Microbacterium testaceum | AP012052.1 |
| Microbacterium sp. | AP017975.1 |
| Pseudomonas sp. | LT707063.1 |
| Pantoea agglomerans | CP016889.1 |
| Pseudomonas sp. | LT707063.1 |
| Pseudomonas mandelii | CP005960.1 |
| Sphingomonas sp. | CP023705.1 |
| Sphingomonas melonis | CP023705.1 |
| Serratia proteamaculans | CP000826.1 |

In some aspects, the microbial inoculant compositions further comprise one or more of yeast strain TAH3020 or yeast strain TAH3021.

The microbial inoculant composition can promote plant growth (e.g., increase leaf size, increase root mass), decrease the impact of stress, decrease water consumption, increase solubility and/or assimilation of nutrients, increase feed value, increase decay of carbon-containing molecules so that the organic molecules are more readily available to the plant, increase production of hormones in plants, and/or increase plant metabolism (thereby decreasing the time to fruit). Moreover, in legumes, the microbial inoculant composition can increase pod numbers, increase root growth, increase nodulation, and/or increase the number of branches per plant. In some aspects, the microbial inoculant composition can be applied to contact and/or interact endophytically with the plant.

In some aspects, bacteria in the microbial inoculant composition can produce 1-aminocyclopropane-1-carboxylate (ACC) deaminase. ACC can lower plant ethylene levels, often a result of various stresses such as, for example, stress to heat and/or drought. ACC can interact synergistically with the plant and bacterial auxin, indole-3-acetic acid (IAA). ACC-producing bacteria not only can directly promote plant growth, but also can protect plants against flooding, drought, salt, flower wilting metals, organic contaminants, bacterial pathogens, and fungal pathogens.

In some aspects, decreasing water consumption can increase solubilization of minerals and/or fertilizers so that water requirements are reduced to transport the minerals and/or fertilizers from the roots, increase root development so that soil nutrients can be obtained from a greater area and/or water can be obtained from deeper in the soil, and/or reduce daily heat stress. Reducing daily heat stress allows the plant to better acquire CO₂, thereby metabolize more sugars and increase yield, regulate pH, and/or produce more energy during daylight hours.

The microbial inoculant compositions can include additional microbial species or other additives to induce the plant to perform desired physiological, metabolic, or other activity. For example, in some aspects, the microbial inoculant compositions can include one or more of the following microbial species: an Acetobacteraceae, spp. (e.g., Acidisphaera spp.), an Acetivibrio spp. (e.g., Acetivibrio cellulolyticus), an Acidiphilium spp., an Acidimicrobiaceae spp. (e.g., an Acidimicrobium spp., an Aciditerrimonas spp.), an Acidobacteriales spp. (e.g., an Acidobacteriaceae spp. [e.g., an Acidobacterium spp.]), an Acidothermus spp., an Acidovorax spp. (e.g., Acidovorax citrulli), an Acinetobacter spp. (e.g., Acinetobacter lwoffii), an Actinoallomurus spp. (e.g., Actinoallomurus iriomotensis), an Actinocatenispora spp. (e.g., Actinocatenispora rupis), an Actinomadura spp., an Actinomycetales spp. (e.g., an Actinomyces spp.), an Actinoplanes spp. (e.g., Actinoplanes auranticolor), an Actinopolymorpha spp. (e.g., Actinopolymorpha pittospori), an Actinotalea spp. (e.g., Actinotalea fermentans), an Adhaeribacter spp. (e.g., Adhaeribacter terreus), an Aeromicrobium spp. (e.g., Aeromicrobium fastidiosum), an Afipia spp., an Agromyces spp. (e.g., Agromyces ulmi, Agromyces subbeticus), an Alcaligenaceae spp., an Algoriphagus spp., an Alkaliflexus spp., an Alphaproteobacteria spp., an Alsobacter spp. (e.g., Alsobacter metallidurans), an Altererythrobacter spp., an Alteromonadaceae spp., an Amaricoccus spp., an Aminobacter spp., an Amycolatopsis spp. (e.g., Amycolatopsis iriomotensis, Amycolatopsis vancoresmycina), an Anaeromyxobacteraceae spp. (e.g., an Anaeromyxobacter spp. [e.g., Anaeromyxobacter dehalogenans]), an Ancylobacter spp., an Angustibacter spp. (e.g., Angustibacter peucedani), an Aquabacterium spp., an Aquicella spp., an Armatimonadetes spp., an Arenimonas spp. (e.g., Arenimonas oryziterrae), an Arsenicicoccus spp. (e.g., Arsenicicoccus dermatophilus), an Arthrobacter spp. (e.g., Arthrobacter pascens, Arthrobacter tumbae), an Asanoa spp. (e.g., Asanoa ishikariensis), an Azohydromonas spp. (e.g., Azohydromonas australica), an Azonexus spp., an Azospira spp. (e.g., Azospira oryzae), an Azospirillum spp. (e.g., Azospirillum lipoferum), an Azotobacter spp. (e.g., Azotobacter chroococcum), a Bacillaceae spp. (e.g., a Bacillus spp. [e.g., Bacillus acidiceler, Bacillus aphidicola, Bacillus senegalensis, Bacillus megaterium, Bacillus subtilis]), a Bacteroidetes spp. (e.g., a Bacteroidales spp. [e.g., a Bacteroides spp.]), a Bauldia spp. (e.g., Bauldia consociate), a Bdellovibrionaceae spp., a Beijerinckia spp., a Blastococcus spp. (e.g., Blastococcus saxobsidens), a Blastomonas spp., a Bordetella spp. (e.g., Bordetella hinzii), a Bosea spp., a Bradyrhizobiaceae, spp. (e.g., Bradyrhizobium spp. [e.g., Bradyrhizobium elkanii, Bradyrhizobium yuanmingense]), a Brevibacteriaceae spp., a Brevundimonas spp. (e.g., Brevundimonas lenta), a Bryobacter spp., a Burkholderiales spp. (e.g., a Burkholderiaceae spp. [e.g., a Burkholderia spp.]), a Brucellaceae spp., a Buttiauxella spp. (e.g., Buttiauxella izardii), a Byssovorax, spp., a Caldilineales spp. (e.g., a Caldilineaceae spp. [e.g., a Caldilinea spp.]), a Caloramator spp., a Candidatus spp. (e.g., Candidatus brocadiaceae, Candidatus entotheonella, Candidatus koribacter, Candidatus nitrosoarchaeum, Candidatus phytoplasma, Candidatus saccharibacteria, Candidatus solibacter), a Carnobacterium spp., a Catenuloplanes spp., a Catellatospora spp., (e.g., Catellatospora citrea), a Caulobacteraceae spp. (e.g., a Caulobacter spp. [e.g., Caulobacter tundrae]), a Cellulosimicrobium spp. (e.g., Cellulosimicrobium cellulans), a Cellvibrio spp. (e.g., Cellvibrio vulgaris), a Cellulomonas spp. (e.g., Cellulomonas terrae), a Chelatococcus spp. (e.g., Chelatococcus asaccharovorans, a Chitinophagaceae spp., a Chromobacteriaceae spp., a Chloroflexales spp. (e.g., a Chloroflexaceae spp. [e.g., a Chloroflexus spp.]), a Chthoniobacter spp. (e.g., Chthoniobacter flavus), a Chryseobacterium spp., a Citrobacter spp., a Clavibacter spp. (e.g., Clavibacter michiganensis), a Clostridiaceae spp. (e.g., a Clostridium spp. [e.g., Clostridium bowmanii, Clostridium gasigenes, Clostridium uliginosum, Clostridium vincentii]), a Comamonadaceae spp. (e.g., a Comamonas, spp. [e.g., Comamonas koreensis]), a Conexibacteraceae spp. (e.g., a Conexibacter spp. [e.g., Conexibacter woesei]), a Coxiellaceae spp., a Crenotrichaceae spp. a Cryomorphaceae spp., a Cryobacterium spp. (e.g., Cryobacterium mesophilum), a Cupriavidus spp. (e.g., Cupriavidus campinensis), a Curtobacterium spp., a Cyanobacteria spp., a Cyclobacteriaceae spp., a Cystobacteraceae spp. (e.g., a Cystobacter spp.), a Cytophagaceae spp. (e.g., a Cytophaga spp.), a Defluviicoccus spp., a Dehalococcoidales spp. (e.g., a Dehalogenimonas spp., a Dehalococcoides spp.), a Denitratisoma spp., a Derxia spp., a Desulfovibrionales spp. (e.g., a Desulfobacteraceae spp. [e.g., a Desulfocapsa spp., a Desulfatiglans spp., a Desulforegula spp.]), a Desulfoglaeba spp., a Desulfosporosinus spp. (e.g., Desulfosporosinus meridiei), a Desulfotomaculum spp., a Desulfuromonadales spp. (e.g., a Desulfuromonas spp.), a Devosia spp. (e.g., Devosia insulae), a Dickeya spp. (e.g., Dickeya zeae), a Dyadobacter spp., an Ectothiorhodospiraceae spp., an Elusimicrobia spp. (e.g., an Elusimicrobiaceae spp. [e.g., an Elusimicrobium spp.]), an Endomicrobia spp., an Enhygromyxa spp. (e.g., Enhygromyxa salina), an Epilithonimonas spp., an Erwinia spp. (e.g., Erwinia persicina), an Exiguobacterium spp. (e.g., Exiguobacterium undae), a Ferrimicrobium spp., a Fictibacillus spp., a Flavobacteriales spp. (e.g., a Flavobacteriaceae, [e.g., a Flavobacterium spp. such as, for example, Flavobacterium arsenatis, Flavobacterium columnare, Flavobacterium hauense, Flavobacterium johnsoniae, Flavobacterium terrigena]), a Flavisolibacter spp., a Flexibacter spp., a Flindersiella spp., a Fodinicola spp., a Frankia spp., Frigoribacterium spp., a Gaiellales spp. (e.g., a Gaiella spp. [e.g., Gaiella occulta]), a Gallionellaceae spp. (e.g., a Gallionella spp.), a Gemmatimonadales spp. (e.g., a Gemmatimonadaceae spp. [a Gemmatimonas spp.]), a Gemmata spp., a Geoalkalibacter spp., a Geobacillus spp., a Geobacteraceae spp. (e.g., a Geobacter spp.), a Gillisia spp., a Glycomyces spp. (e.g., Glycomyces harbinensis), a Halomonas spp. (e.g., Halomonas muralis), a Haliangium spp., a Herbaspirillum spp. (e.g., Herbaspirillum huttiense), a Holophagales spp. (e.g., a Holophagaceae spp. [e.g., a Holophaga spp.]), a Humibacillus spp. (e.g., Humibacillus xanthopallidus), a Hydrogenophaga spp. (e.g., Hydrogenophaga palleronii), a Hydrogenophilaceae spp., a Hyphomicrobiaceae spp. (e.g., a Hyphomicrobium spp. [e.g., Hyphomicrobium methylovorum]), a Hyphomonas spp., an Iamiaceae spp. (e.g., an Iamia spp.), an Ideonella spp., an Ignavibacteriales spp. (e.g., an Ignavibacteriaceae spp. such as, for example, an Ignavibacterium spp.), an Ilumatobacter spp., an Intrasporangiaceae spp. (e.g., an Intrasporangium spp. [e.g., Intrasporangium oryzae]), a Jiangella spp., a Kaistia spp., a Kaistobacter spp., a Kallotenuales spp., a Kineococcus spp., a Kineosporia spp. (e.g., Kineosporia mikuniensis), a Knoellia spp., a Kofleriaceae spp. (e.g., a Kofleria spp.), a Kribbella spp. (e.g., Kribbella karoonensis, Kribbella swartbergensis), a Labedella spp., a Labilitrichaceae spp. (e.g., a Labilithrix spp. [e.g., Labilithrix luteola]), a Lactobacillus spp., a Lactococcus spp. (e.g., Lactococcus garvieae), a Lapillicoccus spp. (e.g., Lapillicoccus jejuensis), a Legionellaceae spp., a Leifsonia spp., a Lentzea spp. (e.g., Lentzea albida), a Leptospira spp., a Leptothrix spp., a Leucobacter spp. (e.g., Leucobacter tardus), a Longilinea spp., a Lysinibacillus spp. (e.g., Lysinibacillus sphaericus), a Lysobacter spp., a Marinimicrobium spp., a Marinobacter spp., a Marmoricola spp., a Massilia spp. (e.g., Massilia timonae), a Melioribacteraceae spp. (e.g., a Melioribacter spp.), a Mesorhizobium spp. (e.g., Mesorhizobium loti, Mesorhizobium plurifarium), a Methylibium spp., a Methylobacillus spp. (e.g., Methylobacillus flagellates), a Methylobacteriaceae spp. (e.g., a Methylobacterium spp. [e.g., Methylobacterium adhaesivum]), a Methylocella spp., a Methylococcaceae spp. (e.g., a Methylobacter spp.), a Methylocystaceae spp. (e.g., a Methylocystis spp. [e.g., Methylocystis echinoides]), a Methylosinus spp., a Methyloversatilis spp., a Microbacteriaceae spp. (e.g., a Microbacterium spp. [e.g., Microbacterium kitamiense], a Microcella spp. [e.g., Microcella alkaliphile]), a Micrococcaceae spp., a Microlunatus spp., a Microvirga spp. (e.g., Microvirga aerilata, Microvirga subterranean), a Mycobacteriaceae spp. (e.g., a Mycobacterium spp. [e.g., Mycobacterium sacrum, Mycobacterium salmoniphilum, Mycobacterium septicum]), a Micromonosporaceae spp. (e.g., a Micromonospora spp. [e.g., Micromonospora rhodorangea]), a Modestobacter spp. (e.g., Modestobacter multiseptatus), a Moorella spp., a Myxococcales spp., a Nakamurella spp., a Nannocystaceae spp. (e.g., a Nannocystis spp. [e.g., Nannocystis exedens]), a Neorhizobium spp. (e.g., Neorhizobium huautlense), a Niastella spp., a Nitriliruptor spp., a Nitrosomonadaceae spp. (e.g., a Nitrosomonas spp. [e.g., Nitrosomonas communis, Nitrosomonas ureae]), a Nitrosopumilales spp. (e.g., a Nitrosopumilaceae spp.), a Nitrosospira spp., a Nitrosovibrio spp. (e.g., Nitrosovibrio tenuis), a Nitrospirales spp. (e.g., a Nitrospira spp.), a Nocardiaceae spp. (e.g., a Nocardia spp. [e.g., Nocardia anaemiae]), a Nocardioidaceae spp. (e.g., a Nocardioides spp. [e.g., Nocardioides albus, Nocardioides iriomotensis, Nocardioides islandensis, Nocardioides maritimus, Nocardioides perillae, Nocardia pneumoniae]), a Nocardiopsis spp. (e.g., Nocardiopsis synnemataformans), a Nonomuraea spp. (e.g., Nonomuraea kuesteri), a Nordella spp., a Novosphingobium spp., an Ochrobactrum spp. (e.g., Ochrobactrum haematophilum), an Ohtaekwangia spp., an Olivibacter spp. (e.g., Olivibacter soli), an Opitutaceae spp., an Oryzihumus spp., an Oxalobacteraceae spp., an Oxalophagus spp. (e.g., Oxalophagus oxalicus), a Paenibacillus spp., (e.g., Paenibacillus graminis, Paenibacillus chondroitinus, Paenibacillus validus), a Pantoea spp. (e.g., Pantoea agglomerans), a Paracoccus spp., a Paracraurococcus spp., a Parastreptomyces spp., a Pasteuriaceae spp., (e.g., a Pasteuria spp.), a Pedosphaera spp. (e.g., Pedosphaera parvula), a Pedobacter spp. (e.g., Pedobacter tournemirensis, Pedobacter kribbensis, Pedobacter kwangyangensis), a Pelagibacterium spp. (e.g., Pelagibacterium halotolerans), a Pelobacteraceae spp. (e.g., a Pelobacter spp.), a Peptoclostridium spp. (e.g., Peptoclostridium clostridium sordellii), a Peredibacter spp., a Phaselicystidaceae spp., a Phenylobacterium spp., a Phycicoccus spp., a Phycisphaerae spp., a Phyllobacterium spp. (e.g., Phyllobacterium trifolii), a Pigmentiphaga spp., a Planococcus spp., a Planomicrobium spp., (e.g., Planomicrobium novatatis), a Planctomycetes spp. (e.g., a Pirellula spp., such as Pirella staleyi), a Plesiocystis spp., a Polaromonas spp., a Polyangiaceae spp., a Procabacteriacae spp., a Prolixibacter spp., a Promicromonospora spp., (e.g., Promicromonospora sukumoe), a Prosthecobacter spp., a Prosthecomicrobium spp., a Pseudoalteromonas spp., a Pseudoclavibacter spp., (Pseudoclavibacter helvolus), a Pseudolabrys spp., (e.g., Pseudolabrys taiwanensis), a Pseudomonadaceae spp. (e.g., Pseudomonas fluorescens, Pseudomonas flavescens, Pseudomonas protegens, Pseudomonas veronii, Pseudomonas rhodesiae, Pseudomonas koreensis, Pseudomonas moorei, Pseudomonas baetica), a Pseudonocardia spp., (e.g., Pseudonocardia zijingensis, Pseudonocardia carboxydivorans), a Pseudorhodoferax spp., a Pseudoxanthobacter spp., a Pseudoxanthomonas spp., a Ralstonia spp., a Ramlibacter spp., a Reyranella spp. (e.g., Reyranella massiliensis), a Rheinheimera spp., a Rhizobiales spp. (e.g., a Rhizobiaceae spp., a Rhodobiaceae spp.), a Rhizobium spp. (e.g., Rhizobium etli), a Rhizomicrobium spp., a Rhodobacterales spp. (e.g., a Rhodobacter spp.), a Rhodococcus spp. (e.g., Rhodococcus gordoniae, Rhodococcus kroppenstedtii, Rhodococcus wratislaviensis), a Rhodocyclales spp. (e.g., a Rhodocyclaceae spp.), a Rhodomicrobium spp., a Rhodoplanes spp. (e.g., Rhodoplanes elegans), a Rhodopseudomonas spp., a Rhodospirillales spp. (e.g., a Rhodospirillaceae spp.), a Rhodothermus spp., a Rickettsiaceae spp., a Roseateles spp., a Roseomonas spp., a Rubrivivax spp. (e.g., Rubrivivax gelatinosus), a Rubrobacterales spp. (e.g., a Rubrobacter spp.), a Ruminococcaceae spp., a Saccharopolyspora spp. (e.g., Saccharopolyspora gloriosa), a Sandaracinus spp., a Saprospiraceae spp., a Serratia spp. (e.g., Serratia proteamaculans), a Shimazuella spp. (e.g., Shimazuella kribbensis), a Shinella spp. (e.g., Shinella granuli), a Sideroxydans spp. (e.g., Sideroxydans lithotrophicus, Sideroxydans paludicola), a Sinobacteraceae spp. (e.g., a Steroidobacter spp.), a Sinorhizobium spp., a Solibacteraceae spp. (e.g., a Solibacter spp.), a Solirubrobacteraceae spp. (e.g., a Solirubrobacter spp.), a Sorangium spp. (e.g., Sorangium cellulosum), a Sphaerobacterales spp. (e.g., a Sphaerobacteraceae spp. such as, for example, a Sphaerobacter spp.), a Sphingobacteriales spp. (e.g., a Sphingobacteriaceae spp. such as, for example, a Sphingobacterium spp.), a Sphingobium spp. (e.g., Sphingobium herbicidovorans), a Sphingomonadaceae spp. (e.g., a Sphingobium spp. [e.g., S. xenophagum], a Sphingomonas spp. [e.g., S. wittichii]), a Sphingopyxis spp. (e.g., Sphingopyxis macrogoltabida), a Sphingosinicella spp., a Spirochaetales spp. (e.g., a Spirochaeta spp.), a Sporichthyaceae spp. (e.g., a Sporichthya spp.), a Stackebrandtia spp. (e.g., Stackebrandtia nassauensis, a Stella spp., a Stenotrophomonas spp. (e.g., Stenotrophomonas maltophilia), a Stigmatella spp. (e.g., Stigmatella erecta), a Streptacidiphilus spp., a Streptoalloteichus spp., a Streptomycetaceae spp. (e.g., a Streptomyces spp. [e.g., Streptomyces aculeolatus, Streptomyces clavuligerus, Streptomyces fradiae, Streptomyces ghanaensis, Streptomyces glauciniger, Streptomyces hebeiensis, Streptomyces heteromorphus, Streptomyces mashuensis, Streptomyces microflavus, Streptomyces netropsis, Streptomyces phaeochromogenes, Streptomyces roseogriseolus, Streptomyces variabilis, Streptomyces vayuensis, Streptomyces viridodiastaticus, Streptomyces viridochromogenes, Streptomyces xylophagus, Streptomyces xinghaiensis]), a Sulfuricella spp., a Syntrophobacterales spp. (e.g., a Syntrophorhabdaceae spp. such as, for example, a Syntrophobacter spp. [e.g., S. wolinii], a Syntrophorhabdus spp., a Syntrophaceae spp., a Syntrophus spp.), a Taibaiella spp., a Tepidamorphus spp., a Terrabacter spp., a Terriglobus spp., a Terrimonas spp., a Tetrasphaera spp. (e.g., Tetrasphaera elongate), a Thermoanaerobacterales spp. (e.g., a Thermoanaerobacteraceae spp.), a Thermoflavimicrobium spp., a Thermoleophilaceae spp., a Thermomonosporaceae spp., a Thioalkalivibrio spp., a Thiobacillus spp., (e.g., Thiobacillus denitrificans), a Thiobacter spp., a Thiomonas spp., a Thiorhodovibrio spp., a Tolumonas spp., (e.g., Tolumonas auensis) a Variovorax spp., (e.g., Variovorax paradoxus), a Verrucomicrobiales spp., (e.g., a Verrucomicrobia subdivision 3 spp.), a Vibrionales spp., a Woodsholea spp., (e.g., Woodsholea maritima), a Xanthomonadaceae spp., (e.g., a Xanthomonas spp.), a Zoogloea spp., or a Zooshikella spp.

In some aspects, the following can act as an antagonist to at least one of the microbial species listed above, e.g., such as Pseudomonas fluorescens, Pseudomonas Streptornyces hygroscopicus, Mycobacterium vaccae, Agrobacterium turnefaciens, Bacillus megaterium, Bacillus amyloliquifaciens, Bacillus subtilus, Bacillus pumilus, a Shingomonas spp., Sphingomonas melonis, an Arthrobacter spp., Agrobacterium rhizogenes, Serratia proteatnaculans Microbacterium testaceum, a Pseudomonas spp., an Erwinia spp., Pantoea agglomerans, Pseudornonas inandelii, a Microbacterium spp., Clostridium saccharobutylicum, Pseudomonas moraviensis, Pantoea vagans, Serratia liquefaciens, Pedobacter kribbensis, Tolumonas auensis, Janthinobacterium lividum, Bacillus racemilacticus, Sporolactobacillus laevolacticus, Brevundimonas mediterranea, Pantoea cloacae, Clostridium acidisoli, Erwinia aphidicola, Bacillus arbutinivorans, Paenibacillus grarninis Pseudomonas veronii, Pseudomonas rhodesiae, Pseudornonas koreensis, Tolumonas auensis, Pseudomonas moorei, Pseudomonas baetica, and/or Pseudomonas protegens.

In some aspects, a microbial species that provides insecticidal activity can be added to the microbial inoculant. Suitable microbes can include bacteria or fungi that produce phytochemicals that have insecticidal or insect repelling properties. In some aspects, the microbial species can be a bacterium such as, for example, B. thuringiensis, B. pipilliae, Photohabdus luminescens, Pseudomonas entomohpilia, Envinia aphidicola, etc., or a fungus such as, for example, Beaveria bassiana, Lagenidium giganteum, etc.

The microbial inoculant composition also can include one or more non-microbial additives. For example, the microbial inoculant composition can include one or more macronutrients or one or more micronutrients such as, for example, carbon, nitrogen, potassium, phosphorus, zinc, magnesium, selenium, chromium, tin, manganese, cobalt, zinc, and/or copper.

Suitable macronutrients or micronutrients may enhance the longevity of the bacteria and microbes leading to a longer shelf life. Also, adding a slow growth supporting carbon source (e.g., glycerol, a vegetable oil, lignin, etc.) may be beneficial. This can also function as a stratification media for more anaerobic and aerobic microbes in a single package.

In some aspects, the microbial inoculant composition can include one or more plant hormones such as, for example, an auxin. Exemplary suitable plant hormones include but are not limited to auxins such as indole-3-acetic acid (IAA), 4-chloroindole-3-acetic acid (4-CI-IAA), 2-phenylacetic acid (PAA), indole-3-butyric acid (IBA), indole-3-propionic acid (IPA), naphthaleneacetic acid (NAA). Adding a plant hormone to the inoculant composition can provide an initial boost of plant growth and/or establish a faster growth pattern in a field that has, for example, sustained crop damage and is replanted so that the replanted crops need to mature faster than usual.

In some aspects, the microbial inoculant composition can include a fertilizing agent. A fertilizing agent may include an organic fertilizing agent or an inorganic fertilizing agent. Exemplary inorganic fertilizing agents may include, for example, nitrogen, phosphorus, potassium, zinc, and/or magnesium. Exemplary organic fertilizers may include, for example, compost, manure, agricultural waste, bone meal, humic extract of peat, and the like or other as known by persons skilled in the art.

In some aspects, the microbial inoculant composition can include one or more adhesive agents to promote the composition adhering to a plant once it is applied to a plant or crop field. In some aspects, the adhesive agent can include any biocompatible adhesive agent that can be mixed with the microbial inoculant composition and dried onto a seed. As used herein, “biocompatible” refers to an agent that is compatible with the other components of the composition, and not deleterious to the seed or plant to which a formulation that includes the biocompatible component is applied. Suitable adhesive agents include talc, graphite, gum agar, cane sugar, dextrin, commercial potato shellac, starch, or other as known by persons skilled in the art.

In some aspects, this disclosure describes a plant to which any embodiment of the microbial inoculant composition described above is applied. Suitable plants include but are not limited to terrestrial plants, such as, for example, crop plants, trees (deciduous or coniferous), feed plants (e.g., alfalfa), biomass crops, or horticultural plants.

Exemplary crop plants can include wheat, oats, barley, cotton, sugar beets, flax, peanuts, beans, soy beans, potatoes, tomatoes, peppers, corn (especially following sugar beet syndrome), cucumbers, lettuce, cabbage, cauliflower, broccoli, radishes, carrots, celery, jalapeno peppers, okra, Brussels sprouts, watermelon, musk melon, apples, pears, grapes, peaches, oranges, grapefruit, plums, apricots, lemons, avocados, bananas, cassava, sweet potato, pineapple, dates, figs, almonds, walnuts, hazel nuts, pecans, cashews, tobacco, cannabis, oregano, cilantro, sage, saffron, cinnamon, agave, other herbs, or other as known by persons skilled in the art.

Exemplary biomass crop plants can include, poplar trees, switch grass, duck weed, elephant grass, moringa, or other as known by persons skilled in the art.

Exemplary trees to which any embodiment of the microbial inoculant composition can be applied include, for example, cottonwood, willow, birch, poplar, or other as known by persons skilled in the art.

Exemplary horticultural plants can include roses, vines, tubered perennials, petunias, hollyhocks, daffodils, reed sedge, tulips, chrysanthemums, or other as known by persons skilled in the art.

For example, when applied to wheat, the microbial inoculant composition can result in increased stem count, increased tillering, increased head weights, increased seed count, increased size of leaves, increased kernel count, increased kernel weight, increased protein content in the kernel, increased height of the stem, and/or increased overall surface area of the flag leaf. In one example, untreated wheat yielded approximately 50 bushels per acre. A comparable field was treated with a microbial inoculant composition at the foliar stage, yield was increased to 75 bushels per acre. A comparable field treated at the seed coat stage yielded more than 100 bushels per acre. The wheat treated at the seed coat stage had a 30% increase in the number of kernels, a 20% increase in kernel weight, and a 2% increase in the ratio of protein in the kernel.

The effect of the microbial inoculant composition can be mitigated to some extent if used in combination with certain fungicides such as, for example, propiconazole. If the fungicide is applied at the manufacturer recommended rate, the efficacy of the microbial inoculant composition can be reduced. For example, when applied to wheat before jointing, the fungicide kills bacteria in the microbial inoculant composition and the effects of the microbial inoculant composition can be negated. If the fungicide is applied to wheat after jointing, one can still see an increase in head count, but increases in leaf size, kernel size, protein ratio, etc. are mitigated.

When applied to soybeans, the microbial inoculant composition can result in, for example, increased branching, increased pod count, increased leaf count, increased leaf size, increased number of root nodules, and/or increased size of root nodules. In at least one embodiment, the microbial inoculant composition can be applied at an end of a vegetative state of the soybeans. Results of applying the microbial inoculant composition to soy beans can include an increase of anywhere from 4 to 8 bushels per acre. In at least one example result, one field had an increase of 16 bushels per acre. In at least one example method, the microbial inoculant composition is applied to the seed coat, an herbicide is added to damage the leaves of the plant, a Hydra effect occurs, additional herbicide is added to the leaves, and the stalks are broken to further induce the Hydra effect.

When applied to potatoes, the microbial inoculant composition can result in, for example, increased early stage rooting, increased rhizome production, increase the weight of salable potatoes by promoting the first and second set over the third and fourth set, produce darker coloration, increase the above-ground mass of the plant, and/or increase the total weight of tubers produced per acre. In at least one example, the microbial inoculant composition can be applied to potatoes and/or rooted plants, such as sugar beets, onions, carrots, etc. In at least one example of application to onions, a single onion can grow to approximately 3.25 lbs. In contrast, an onion that has not received the microbial inoculant composition can grow to about 0.25 to 0.5 lbs. In addition, in at least one example, onions with the application can have increased volume with less time to get to the onion's normal size, mentioned above. In at least one example, application of the microbial inoculant composition on sugar beets, without splitting, can result in a weight increase of 300%. In at least one example, application of the microbial inoculant composition on sweet potatoes can result in a two-fold increase in size of the sweet potato.

When applied to trees, the microbial inoculant composition can result in, for example, increased height, increased number of leaves in the first year, and/or increased total mass of the tree.

When applied to tomatoes, the microbial inoculant composition can result in, for example, increased flowering, increased bud count, better regeneration after browsing, and/or increased number of tomatoes produced per plant.

When applied to alfalfa, the microbial inoculant composition can result in, for example, increased volume of plant material per acre and/or reduced effects of stress flowering. Reducing the effects of stress flowering allows one to wait longer to cut the alfalfa before it turns woody. In spring, this can allow a farmer to allow the alfalfa to grow longer before it turns woody, thereby allowing the farmer to spend time planting other crops that would otherwise be necessary to cut the alfalfa before it turns woody. Waiting longer between cuttings before the alfalfa turns woody allows one to obtain more tonnage without sacrificing the quality and/or nutritional value of the alfalfa. Also, applying the microbial inoculant composition to alfalfa can result a decrease in the lignin content of the plant as a percentage of total plant biomass. The decreased lignin content can increase the food value of the plant. Applying the microbial inoculant composition also can increase leaf size and/or increase root mass of the plant. Increasing leaf size, like decreasing the lignin content, can increase the food value of the plant. To support the increased photosynthetic surface area that results from the increased leaf size, pants treated with the microbial inoculant composition can exhibit increased root mass, thereby increasing the carbon in the soil. When applied to alfalfa, it may be desirable to reapply the microbial inoculant composition after each cutting.

In some aspects, in response to applying the microbial inoculant composition, alfalfa production can increase by 15 percent in alfalfa production by tonnage. In at least one embodiment, a Rhizobium species and/or minerals including cobalt can be added along with or be added within the microbial inoculant composition. In at least one example, inoculation of alfalfa occurred two weeks prior to cutting, resulting in a 35% increase in tonnage.

The effects of the microbial inoculant composition on alfalfa can be reduced somewhat when there is a zinc deficiency and/or molybdenum deficiency in the soil and/or alfalfa, such as may occur when alfalfa is repeatedly grown in the same field. The mineral deficiency can become a growth-limiting factor. The mineral deficiency can affect the activity of indole-3-acetic acid (IAA) and other growth hormones, affecting the ability of the plant to convert nitrate to ammonium.

When applied to sunflowers, the microbial inoculant composition can result in, for example, increased surface area of flower heads, increased sugars in the flowers, and/or a Hydra effect. In at least one embodiment, a greater than or equal to increase in surface area of flower heads was observed. Increased sugars in the flowers can increase attraction of pollinators and, therefore, increase pollination. The microbial inoculant composition can be added to the sunflower plants in response to the flower heads being at least 3 inches tall, just post-emergence.

In at least one example, a Hydra effect including cutting off a first head and growing two replacement heads that are full heads 10½ inches tall was observed. In this example, this can double the yield of sunflower heads.

When applied to bell peppers, the microbial inoculant composition can result in, for example, increased weight of the fruit, increased stem rigidity, and/or increased stem strength.

When applied to corn, the microbial inoculant composition can result in, for example, increased number of kernels per ring and/or increased phosphorus solubility for the plant, thereby mitigating effects of sugar beet syndrome in which an untreated corn plant can manifest stunted plant growth, decreased yield, and/or the corn having a purple appearance. In at least one embodiment, in response to the microbial inoculant composition being applied to corn, a yield increase of one ton to 2.5 tons per acre of dry land silage can result. The application of the microbial inoculant composition is not time dependent; the microbial inoculant composition can be applied at any time from VI to tassel. When applied to grain corn, in at least one embodiment, within a week of the tassels a 4.8 to 6.8 bushel per acre yield increase can result. In at least one example where corn following sugar beet (CFS) syndrome has occurred, application of the microbial inoculant composition at seed coat or at post-emergence can stabilize phosphorus, leading to the corn overcoming the CFS syndrome effects. CFS syndrome can refer to when corn planting directly follows the planting of sugar beets, which can lead to stunting, shortened internodes, purpling, and/or reduction in vigor.

When applied to small grains, such as wheat barley, oats, rye, etc., applying the microbial inoculant composition prior to a flag leaf can increase the size of the flag leaf, which can, in turn, increase the supply of carbohydrates available to feed the grains. That is, the mass of the small grain can be increased, which can increase tonnage of the small grains. In at least one example, early application prior to a tiller (e.g., stem) and flag leaf can increase a quantity of stems and increase the weight of the small grain, increasing the tonnage by from 50% to as much as 100%.

Also, when applied to the seed coat of small grains, the microbial inoculant composition can increase head count. In at least one example, the microbial inoculant composition can be applied rye or winter wheat in the fall season and again in the spring season.

When applied to cabbage, the microbial inoculant composition can include at least one or more of B. thuringiensis and B. amyloliquifaciens. In at least one example, in response to harvesting cabbage plants that received application of the microbial inoculant composition, the cabbage plants produced multiple heads per plant. In contrast, cabbage plants that did not receive application of the microbial inoculant composition died post-harvest.

When applied to grass, such as prairie grass, lawn grass, sod, etc., the microbial inoculant composition can be applied to both the seed and the grass, increasing leaf size and promoting a darker color, increased growth, and increased root growth that can capture more carbon and/or store increased amounts of carbon in the soil.

When applied to hemp, the microbial inoculant composition can result in, for example, increased height, increased width, increase root size, increased stem girth, increased number of buds, increased size of buds, increased number of seed structures, and/or increased size of seed structures.

When applied to duckweed, the microbial inoculant composition can result in increased root growth. In at least one example, where duckweed can grow up to approximately one (1) inch, application of the microbial inoculant composition can result in growth up to 12 inches. Further, the increased growth of the duckweed can result in increased phosphotransacetylase (pta) biomass as feed. In at least one example, in response to stressing the duckweed plant (such as with dehydration, heat, pH change, etc.) as it is harvested, a breakdown of leucine can occur. The breakdown of leucine can change the amino acid composition and provide a product with lower or no levels of leucine.

When applied to horticultural plants, the microbial inoculant composition can result in, for example, increased growth (whether measured by height, length, or total mass), increased number of blossoms, deeper coloration, faster growing vine, increased size of vine leaves, increased numbers of runners, increased length of runners, and/or tuber perennials carrying over bacteria from the inoculant to subsequent years. In at least one embodiment, application of the microbial inoculant composition to horticultural plants can maintain turgor pressure longer than plants that where the microbial inoculant composition was not applied, causing the plant to maintain aesthetic appeal longer, which can result in greater retail sales and fewer discarded plants.

In some aspects, post-stress damage can occur to any of the above-mentioned plants, trees, and/or crops. This post-stress damage can include hail damage, wind damaged, flooding, etc. As long as the plant, tree, and/or crop is alive, the more the damage, the greater the response due to the microbial inoculant composition. Results of the response can be seen in as little as two weeks. If the microbial inoculant composition is applied prior to the damage, the regeneration of the plant, tree, and/or crop can occur immediately or in close proximity in time to the damage.

In some aspects, the microbial inoculant composition can be co-fermented. In some aspects, the microbial inoculant composition can comprise a mixture of at least one aerobic species and at least one anaerobic species. During co-fermentation, the aerobic microbes typically grow more quickly than anaerobic microbes at first. Eventually, fermentation by the aerobes depletes the fermentation broth of oxygen and produces CO₂. Depletion of oxygen in the broth promotes growth of the anaerobic microbes, while accumulation of CO₂ in the broth slows growth of the aerobic microbes. In this way, a microbial inoculant composition comprising an aerobic species and an anaerobic species can be prepared in a single co-fermentation. In some aspects, the microbial inoculant composition can be aerated to facilitate growth of the Pseudomonas spp. The microbial inoculant composition may be prepared by incubating the microbes in a suitable culture medium at any suitable temperature. A suitable culture medium can include a carbon source (e.g., cane sugar or sucrose), sufficient white vinegar to adjust the pH of the culture medium to no higher than 7.0 (e.g., no higher than 6.8), iron, and a source of potassium (e.g., potassium nitrate).

The microbes may be incubated at a minimum temperature of at least 5° C., such as, for example, at least 10° C., at least 15° C. at least 20° C., at least 25° C., at least 30° C., or at least 40° C. The microbes may be incubated at a maximum temperature of no more than 50° C., such as, for example, no more than 45° C., no more than 45° C., no more than 40° C., no more than 35° C., or no more than 30° C. The microbes may be incubated at a temperature characterized by any range that includes, as endpoints, any combination of a minimum temperature identified above and any maximum temperature identified above that is greater than the minimum temperature. For example, in some aspects, the microbes may be incubated at a temperature of from 10° C. to 40° C.

The microbial inoculant composition may be prepared by incubating the microbes in a suitable culture medium for a sufficient time to allow growth of both aerobic and anaerobic microbes in the fermentation culture. When a mixture of aerobic microbes and anaerobic microbes are co-fermented, the microbes may be incubated for a minimum of at least 48 hours, such as, for example, at least 72 hours, at least 96 hours, at least 120 hours, at least 144 hours, or at least 168 hours. The microbes may be incubated for a maximum of no more than 240 hours, no more than 216 hours, no more than 192 hours, no more than 168 hours, no more than 144 hours, no more than 120 hours, or no more than 96 hours. The microbes may be incubated for a period characterized by a range having, as endpoints, any combination of a minimum incubation time listed herein and any maximum incubation time listed herein that is greater than the minimum incubation time.

### Methods

Disclosed herein are method of improving traits of agronomic importance in plants. In some aspects, the methods can comprise: (a) contacting a plant element of the plant with a composition comprising a first bacterial strain comprising Clostridium spp. with a 16S nucleic acid sequence that is at least about 97% identical to any one of the Clostridium spp. listed in Table 1 or Table 2 and a second bacterial strain comprising aquatic Pseudomonas spp. with a 16S nucleic acid sequence that is at least about 97% identical to any one of the Pseudomonas spp. listed in Table 1 or Table 2; and (b) growing the plant element comprising the composition comprising the first bacterial strain and the second bacterial strain into the plant. In some aspects, the plant grown from the contacted plant element can have an improvement in the trait of agronomic importance as compared to a plant not contacted with the composition. In some aspects, the trait of agronomic importance can be selected from the group consisting of: germination rate, emergence rate, shoot biomass, root biomass, root length, number of lateral roots, root surface area, seedling root length, seedling shoot length, leaf surface area, sugar holding capacity, sugar content and yield. In some aspects, the trait of agronomic importance can be sugar holding capacity or sugar content. In some aspects, the plant can be a grape or a melon.

In some aspects, the methods of improving traits of agronomic importance in plants, can comprise: (a) contacting a plant element of the plant with a composition comprising one or more of the microbes listed in Table 1, Table 2, Table 3 or Table 4; and (b) growing the plant element comprising the composition comprising one or more of the microbes listed in Table 1. Table 2, Table 3 or Table 4 into the plant. In some aspects, the plant grown from the contacted plant element has an improvement in the trait of agronomic importance as compared to a plant not contacted with the composition. In some aspects, the trait of agronomic importance can be selected from the group consisting of: germination rate, emergence rate, shoot biomass, root biomass, root length, number of lateral roots, root surface area, seedling root length, seedling shoot length, leaf surface area, sugar holding capacity, sugar content and yield. In some aspects, the trait of agronomic importance can be sugar holding capacity or sugar content. In some aspects, the plant can be a grape or a melon.

In some aspects, the methods of improving traits of agronomic importance in plants, the methods can comprise growing the plant from a plant reproductive element that has been contacted with a composition comprising a first bacterial strain comprising Clostridium spp. with a 16S nucleic acid sequence that is at least about 97% identical to any one of the Clostridium spp. listed in Table 1 or Table 2 and a second bacterial strain comprising aquatic Pseudomonas spp. with a 16S nucleic acid sequence that is at least about 97% identical to any one of the Pseudomonas spp. listed in Table 1 or Table 2. In some aspects; the plant grown from the contacted reproductive element can comprise the composition comprising the first bacterial strain and the second bacterial strain. In some aspects, the plant grown from the contacted plant reproductive element can have an improvement in a trait of agronomic importance as compared to an isoline plant grown from a plant reproductive element not contacted with the composition.

In some aspects, the methods of improving traits of agronomic importance in plants, the methods can comprise growing the plant from a plant reproductive element that has been contacted with a composition comprising one or more of the microbes listed in Table 1. Table 2. Table 3 or Table 4. In some aspects, the plant grown from the contacted reproductive element comprises the composition comprising the one or more microbes. In some aspects, the plant grown from the contacted plant reproductive element can have an improvement in a trait of agronomic importance as compared to an isoline plant grown from a plant reproductive element not contacted with the composition.

Disclosed herein are methods of increasing the sugar content of grapes or melons. In some aspects, the methods can comprise: contacting an effective amount of a composition comprising a first bacterial strain and a second bacterial strain with an external surface of the grape or melon. In some aspects, the sugar content of the grape or melon contacted with the composition can have an increased sugar content as compared to a plant not contacted with the composition. In some aspects, a first bacterial strain can comprise Clostridium spp. with a 16S nucleic acid sequence that is at least about 97% identical to any one of the Clostridium spp. listed in Table 1 or Table 2. In some aspects, a second bacterial strain can comprise an aquatic Pseudomonas spp. with a 16S nucleic acid sequence that is at least about 97% identical to any one of the Pseudomonas spp. listed in Table 1 or Table 2.

In some aspects, the methods of increasing the sugar content of grapes or melons can comprise: contacting an effective amount of a composition comprising one or more of the microbes listed in Table 1, Table 2, Table 3 or Table 4 with an external surface of the grape or melon wherein the sugar content of the grape or melon contacted with the composition can have an increased sugar content as compared to a plant not contacted with the composition.

Disclosed herein are methods of increasing the sugar holding capacity of grapes or melons. In some aspects, the methods can comprise: contacting an effective amount of a composition comprising a first bacterial strain and a second bacterial strain with an external surface of the grape or melon. In some aspects, the sugar holding capacity of the grape or melon contacted with the composition can have an increased sugar content as compared to a plant not contacted with the composition. In some aspects, a first bacterial strain can comprise a Clostridium spp. with a 16S nucleic acid sequence that is at least about 97% identical to any one of the Clostridium spp. listed in Table 1 or Table 2. In some aspects, a second bacterial strain comprising an aquatic Pseudomonas spp. with a 16S nucleic acid sequence that is at least about 97% identical to any one of the Pseudomonas spp. listed in Table 1 or Table 2.

In some aspects, the methods of increasing the sugar holding capacity of grapes or melons can comprise: contacting an effective amount of a composition comprising one or more of the microbes listed in Table 1, Table 2, Table 3 or Table 4 with an external surface of the grape or melon. In some aspects, the sugar holding capacity of the grape or melon contacted with the composition can have an increased sugar content as compared to a plant not contacted with the composition.

Disclosed herein are methods of increasing the amount of atmospheric derived nitrogen in plants. In some aspects, the methods can comprise: exposing the plant to a composition comprising a first bacterial strain and a second bacterial strain, and exposing the plant to one or more nitrogen-fixing bacteria thereby increasing an amount of atmospheric derived nitrogen within the plant that is exposed to the composition and the one or more nitrogen-fixing bacteria relative to a plant that is not exposed to the composition. In some aspects, a first bacterial strain can comprise Clostridium spp. with a 16S nucleic acid sequence that is at least about 97% identical to any one of the Clostridium spp. listed in Table 1 or Table 2. In some aspects, a second bacterial strain can comprise aquatic Pseudomonas spp. with a 16S nucleic acid sequence that is at least about 97% identical to any one of the Pseudomonas spp. listed in Table 1 or Table 2. In some aspects, the methods can further comprise exposing or contacting the plant with a nitrogen fixing bacteria.

In some aspects, the methods of increasing the amount of atmospheric derived nitrogen in plants can comprise: exposing the plant to a composition comprising one or more of the microbes listed in Table 1, Table 2, Table 3 or Table 4, and exposing the plant to one or more nitrogen-fixing bacteria thereby increasing an amount of atmospheric derived nitrogen within the plant that is exposed to the composition and the one or more nitrogen-fixing bacteria relative to a plant that is not exposed to the composition. In some aspects, the methods can further comprise exposing or contacting the plant with a nitrogen fixing bacteria.

Disclosed herein are methods of increasing nitrogen production in plants, the methods comprising: (a) contacting a plant element of the plant with a composition comprising a first bacterial strain and a second bacterial strain, and (b) growing the plant element comprising the first bacterial strain and the second bacterial strain into the plant. In some aspects, the plant grown from the contacted plant element can produce more nitrogen as compared to a plant not contacted with the composition. In some aspects, a first bacterial strain can comprise a Clostridium spp. with a 16S nucleic acid sequence that is at least about 97% identical to any one of the Clostridium spp. listed in Table 1 or Table 2. In some aspects, a second bacterial strain can comprise an aquatic Pseudomonas spp. with a 16S nucleic acid sequence that is at least about 97% identical to any one of the Pseudomonas spp. listed in Table 1 or Table 2.

In some methods of increasing nitrogen production in plants, the methods can comprise: (a) contacting a plant element of the plant with a composition comprising one or more of the microbes listed in Table 1, Table 2, Table 3 or Table 4 and (b) growing the plant element comprising the one or more of the microbes into the plant. In some aspects, the plant grown from the contacted plant element can produce more nitrogen as compared to a plant not contacted with the composition.

Nitrogen-fixing bacteria are prokaryotic microorganisms that are capable of transforming nitrogen gas from the atmosphere into “fixed nitrogen” compounds, such as ammonia, that are usable by plants.

Disclosed herein are methods of producing plants. In some aspects, the methods can comprise: applying an isolated bacterial species to a plant, plant seed, or to a growth medium in which the plant is located; culturing the plant under conditions suitable for plant growth; and harvesting the plant. In some aspects, the isolated bacterial species can one or more of the microbes listed in Table 1, Table 2, Table 3 or Table 4. In some aspects, the plant produced comprises one or more of the microbes listed in Table 1, Table 2, Table 3 or Table 4. In some aspects, the methods can comprise: applying an isolated bacterial species to a plant, plant seed, or to a growth medium in which the plant is located; culturing the plant under conditions suitable for plant growth; and harvesting the plant. In some aspects, the isolated bacterial species can be a Clostridium spp.

In some aspects, the methods of producing plants can comprise: applying a composition comprising: a) a purified population of bacteria selected from: (i) Clostridium spp. with a 16S nucleic acid sequence that is at least about 97% identical any of the Clostridium spp. listed in Table 1 or Table 2, (ii) a Pseudomonas spp. with a 16S nucleic acid sequence that is at least about 97% identical to any one of the Pseudomonas spp. listed in Table 1 or Table 2, and/or (iii) a bacteria with a 16S nucleic acid sequence that is at least about 97% identical to any of the bacteria listed in Table 1, Table 2 or Table 3 to a plant, plant seed, or to a growth medium in which the plant is located; culturing the plant under conditions suitable for plant growth; and harvesting the plant. In some aspects, the plant produced comprises the Clostridium spp. In some aspects, the composition can comprise one or more of the microbes listed in Table 1, Table 2, Table 3, or Table 4.

In some aspects, a plant can be selected from the group consisting of: wheat, soy bean, maize, cotton, canola, barley, sorghum, millet, rice, rapeseed, alfalfa, tomato, sugarbeet, sorghum, almond, walnut, apple, peanut, strawberry, lettuce, orange, potato, banana, sugarcane, potato, cassava, mango, guava, palm, onions, olives, peppers, tea, yams, cacao, sunflower, asparagus, carrot, coconut, lemon, lime, barley, watermelon, cabbage, cucumber, grape, and turfgrass.

In some aspects, the amount of the sugar content that is increased can be at least 5% relative prior to administering. In some aspects, the amount of the sugar content that is increased can be between 5% and 99% relative prior to administering. In some aspects, the amount of the sugar content that is increased can be at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or any percent decrease in between relative prior to administering.

In some aspects, the composition described herein can be administered through the ingestion of a feedstock or foodstuff comprising the disclosed compositions. In some aspects, the dose of the composition can be administered such that there exists 10² to 10¹², 10³ to 10¹², 10⁴ to 10¹², 10⁵ to 10¹², 10⁶ to 10¹², 10⁷ to 10¹², 10⁸ to 10¹², 10⁹ to 10¹², 10¹⁰ to 10¹², 10¹¹ to 10¹², 10² to 10¹¹, 10³ to 10¹¹, 10⁴ to 10¹¹, 10⁵ to 10¹¹, 10⁶ to 10¹¹, 10⁷ to 10¹¹, 10⁸ to 10¹¹, 10⁹ to 10¹¹, 10¹⁰ to 10¹¹, 10² to 10¹⁰, 10³ to 10¹⁰, 10⁴ to 10¹⁰, 10⁵ to 10¹⁰, 10⁶ to 10¹⁰, 10⁷ to 10¹⁰, 10⁸ to 10¹⁰, 10⁹ to 10¹⁰, 10² to 10⁹, 10³ to 10⁹, 10⁴ to 10⁹, 10⁵ to 10⁹, 10⁶ to 10⁹, 10⁷ to 10⁹, 10⁸ to 10⁹, 10² to 10⁸, 10² to 10⁸, 10⁴ to 10⁸, 10⁵ to 10⁸, 10⁶ to 10⁸, 10⁷ to 10⁸, 10² to 10⁷, 10³ to 10⁵, 10⁴ to 10⁵, 10² to 10⁴, 10³ to 10⁴, 10² to 10³, 10¹², 10¹¹, 10¹⁰, 10⁹, 10⁸, 10⁷, 10⁶, 10⁵, 10⁴, 10³, or 10² total microbial cells per gram or milliliter of the composition.

In some aspects, the composition can be administered 1 or more times per day. In some aspects, the composition is administered with food each time the animal is fed. In some aspects, the composition can be administered 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, 9 to 10, 1, 2, 3, 4, 5, 6, 7, 8, 9, or 10 times per day.

In some aspects, the composition can be administered 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, 9 to 10, 1, 2, 3, 4, 5, 6, 7, 8, 9, or 10 times per week.

Inn some aspects, the composition can be administered 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, 9 to 10, 1, 2, 3, 4, 5, 6, 7, 8, 9, or 10 times per month.

In some aspects, the composition can be administered 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, 9 to 10, 1, 2, 3, 4, 5, 6, 7, 8, 9, or 10 times per year.

In some aspects, the composition can be administered to animals throughout the entire time they are on the feedlot. In some aspects, the composition can be administered to animals only during a portion of time while they are on the feedlot. In some aspects, the composition can be administered only during the grower phase. In some aspects, the composition can be administered only during the time when animals are in the receiving pen. In some aspects, the composition can administered only when the animals are receiving vaccinations and/or treatments. In some aspects, the composition can administered only when the animals are on a step up diet or when being adapted to a high grain diet. In some aspects, the composition can be administered only when the animals are on a finisher diet or a high grain diet.

In some aspects, the microbial composition can be administered during the grower phase, when animals are in the receiving pen, when animals are receiving vaccinations and/or treatments, when animals are being adapted to a high grain diet or are on a step up diet, and/or when the animals are on a finisher diet or a high grain diet.

In some aspects, an animal entering the feed lot receives at least one composition prior to entering the feed lot. In some aspects, an animal on the feed lot receives a composition that is different from the first at least one composition. In further aspects, an animal on the feed lot receives a composition that is different from the first and second at least one microbial composition.

In some aspects, the type of diet fed to the animal corresponds with the type of composition administered to the animal. In some aspects, a grazing or grass/hay-fed animal will receive a first composition. In further aspects, the same animal fed a different diet will receive a second composition, wherein the first composition can be different from the second composition. In some aspects, the same animal fed yet a different diet will receive a third composition, wherein the first composition can be different from the second and third compositions. In some aspects, the same animal fed yet a different diet will receive a fourth composition, wherein the first composition can be different from the second, third, and fourth compositions. In some aspects, the same animal fed yet a different diet will receive a fifth composition, wherein the first composition is different from the second, third, fourth, and fifth compositions.

In some aspects, the feed can be uniformly coated with one or more layers of the microbes and/or microbial compositions disclosed herein, using conventional methods of mixing, spraying, or a combination thereof through the use of treatment application equipment that is specifically designed and manufactured to accurately, safely, and efficiently apply coatings. Such equipment uses various types of coating technology such as rotary coaters, drum coaters, fluidized bed techniques, spouted beds, rotary mists, or a combination thereof. Liquid treatments such as those of the present disclosure can be applied via either a spinning “atomizer” disk or a spray nozzle, which evenly distributes the microbial composition onto the feed as it moves though the spray pattern. In some aspects, the feed can then be mixed or tumbled for an additional period of time to achieve additional treatment distribution and drying.

In some aspects, the feed coats of the present disclosure can be up to 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, 510 μm, 520 μm, 530 μm, 540 μm, 550 μm, 560 μm, 570 μm, 580 μm, 590 μm, 600 μm, 610 μm, 620 μm, 630 μm, 640 μm, 650 μm, 660 μm, 670 μm, 680 μm, 690 μm, 700 μm, 710 μm, 720 μm, 730 μm, 740 μm, 750 μm, 760 μm, 770 μm, 780μ, 790μ, 800 μm, 810 μm, 820 μm, 830 μm, 840 μm, 850 μm, 860μ, 870 μm, 880 μm, 890 μm, 900 μm, 910 μm, 920 μm, 930 μm, 940 μm, 950 μm, 960 μm, 970 μm, 980 μm, 990 μm, 1000 μm, 1010 μm, 1020 μm, 1030 μm, 1040 μm, 1050 μm, 1060 μm, 1070 μm, 1080 μm, 1090 μm, 1100 μm, 1110 μm, 1120 μm, 1130 μm, 1140 μm, 1150 μm, 1160 μm, 1170 μm, 1180 μm, 1190 μm, 1200 μm, 1210 μm, 1220 μm, 1230 μm, 1240 μm, 1250 μm, 1260 μm, 1270 μm, 1280 μm, 1290 μm, 1300 μm, 1310 μm, 1320 μm, 1330 μm, 1340 μm, 1350 μm, 1360 μm, 1370 μm, 1380 μm, 1390 μm, 1400 μm, 1410 μm, 1420 μm, 1430 μm, 1440 μm, 1450 μm, 1460 μm, 1470 μm, 1480 μm, 1490 μm, 1500 μm, 1510 μm, 1520 μm, 1530 μm, 1540 μm, 1550 μm, 1560 μm, 1570 μm, 1580 μm, 1590 μm, 1600 μm, 1610 μm, 1620 μm, 1630 μm, 1640 μm, 1650 μm, 1660 μm, 1670 μm, 1680 μm, 1690 μm, 1700 μm, 1710μ, 1720 μm, 1730 μm, 1740 μm, 1750 μm, 1760 μm, 1770 μm, 1780 μm, 1790 μm, 1800 μm, 1810 μm, 1820 μm, 1830 μm, 1840 μm, 1850 μm, 1860 μm, 1870 μm, 1880 μm, 1890 μm, 1900 μm, 1910 μm, 1920 μm, 1930 μm, 1940 μm, 1950 μm, 1960 μm, 1970 μm, 1980 μm, 1990 μm, 2000 μm, 2010 μm, 2020 μm, 2030 μm, 2040 μm, 2050 μm, 2060 μm, 2070 μm, 2080 μm, 2090 μm, 2100 μm, 2110 μm, 2120 μm, 2130 μm, 2140 μm, 2150 μm, 2160 μm, 2170 μm, 2180 μm, 2190 μm, 2200 μm, 2210 μm, 2220 μm, 2230 μm, 2240 μm, 2250 μm, 2260 μm, 2270 μm, 2280 μm, 2290 μm, 2300 μm, 2310 μm, 2320 μm, 2330 μm, 2340 μm, 2350 μm, 2360 μm, 2370 μm, 2380 μm, 2390 μm, 2400 μm, 2410 μm, 2420 μm, 2430 μm, 2440 μm, 2450 μm, 2460 μm, 2470 μm, 2480 μm, 2490 μm, 2500 μm, 2510 μm, 2520 μm, 2530 μm, 2540 μm, 2550 μm, 2560 μm, 2570 μm, 2580 μm, 2590 μm, 2600 μm, 2610 μm, 2620 μm, 2630 μm, 2640 μm, 2650 μm, 2660 μm, 2670 μm, 2680 μm, 2690 μm, 2700 μm, 2710 μm, 2720 μm, 2730 μm, 2740 μm, 2750 μm, 2760 μm, 2770 μm, 2780 μm, 2790 μm, 2800 μm, 2810 μm, 2820 μm, 2830 μm, 2840 μm, 2850 μm, 2860 μm, 2870 μm, 2880 μm, 2890 μm, 2900 μm, 2910 μm, 2920 μm, 2930 μm, 2940 μm, 2950 μm, 2960 μm, 2970 μm, 2980 μm, 2990 μm, or 3000 μm thick.

In some aspects, the microbial cells can be coated freely onto any number of compositions or they can be formulated in a liquid or solid composition before being coated onto a composition. For example, a solid composition comprising the microorganisms can be prepared by mixing a solid carrier with a suspension of the spores until the solid carriers are impregnated with the spore or cell suspension. This mixture can then be dried to obtain the desired particles.

In some other aspects, it is contemplated that the solid or liquid compositions of the present disclosure further contain functional agents e.g., activated carbon, minerals, vitamins, and other agents capable of improving the quality of the products or a combination thereof.

Methods of coating and compositions in use of said methods that are known in the art can be particularly useful when they are modified by the addition of one of the embodiments of the present disclosure. Such coating methods and apparatus for their application are disclosed in, for example: U.S. Pat. Nos. 8,097,245 and 7,998,502; and PCT Pat. App. Pub. Nos. WO 2008/076975, WO 2010/138522, WO 2011/094469, WO 2010/111347, and WO 2010/111565 each of which is incorporated by reference herein.

In some aspects, the microbes or microbial compositions of the present disclosure exhibit a synergistic effect, on one or more of the traits described herein, in the presence of one or more of the microbes or microbial compositions coming into contact with one another.

The microbial inoculant may be applied to seeds, plants, or a field of plants by any suitable method. As described above, the microbial inoculant composition may be formulated with a biocompatible adhesive agent that allows the microbial inoculant composition to be applied to, and adhere to, a seed. Such a formulation can be a folair liquid, seed coating, seed coating hydrogel, etc. The formulation can be mixed into a seeder at planting or can be mixed prior to planting. Alternatively, the microbial inoculant composition may be formulated into with one or more biocompatible agents that can be applied to seeds and dried. Suitable agents include but are not limited to, for example, dried tapioca, powdered milk, or gum arabic.

Other application methods can involve applying the microbial inoculant composition to one or more tissues of plant, such as, for example, the root, the stem, one or more leaves, or a seed-producing pod. In such cases, the microbial inoculant composition may be applied by any suitable method including but not limited to, for example, spraying or ampule delivery. The formulation may be sprayed using, for example, a portable spraying unit, hand-held spraying device, irrigation equipment, or aerial spraying. Ampule delivery may be performed manually or using an automated system.

Still other application methods can involve applying the microbial inoculant composition to the soil or seed bed into which seeds will be planted. In some aspects, the microbial inoculant composition may be applied by spraying or ampule delivery as described immediately above. Alternatively, the microbial inoculant composition may be applied by drip. In some aspects, the microbial inoculant composition can be applied, whether by spray or by drip, while the soil is being seeded.

Still other application methods can include application as a foliar spray, through an irrigation pivot, and as a seed coat. In some aspects, a seed coat media that can hold water can be used to allow the bacteria to live without drying out. In some aspects, the bacteria can include primarily non-sporulating bacteria that may die when desiccated.

In some aspects, the methods can include applying the microbial inoculant composition to landfills. The application of the microbial inoculant composition to landfills can be by any suitable method. In some aspects, the application of the microbial inoculant composition to landfills can be in the form of a liquid or a spray.

In some aspects, a formulation of the microbial inoculant composition can comprise a predetermined moisture content. In some aspects, the minimum moisture content can be at least 5% such as, for example, at least 6%, at least 7%, at least 8%, at least 9%, at least 10%, at least 15%, at least 20%, at least 25%, at least 30%, or at least 50%.

In some aspects, a formulation of the microbial inoculant composition can comprise a sugar (e.g., cane sugar or sucrose) and vinegar (e.g., white vinegar). The sugar can provide a metabolic carbon source. The vinegar can provide an acidic pH and/or an alternative carbon source. As an alternative to, or in addition to, the use of vinegar to regulate pH, the microbial inoculant composition can comprise Lactobacillus plantarum, as described herein, to help maintain an acidic pH once the microbial inoculant composition is applied to the plant.

In some aspects, a formulation of the microbial inoculant composition can comprise lactic acid media to provide an acidic pH.

In some aspects, a formulation of the microbial inoculant composition can comprise glycerol as a dispersion medium.

What is claimed is:

1. A method of improving a trait of agronomic importance in a plant, the method comprising: (a) contacting a plant element of the plant with a composition comprising a first bacterial strain comprising Clostridium spp. with a 16S nucleic acid sequence that is at least about 97% identical to any one of the Clostridium spp. listed in Table 1 or Table 2 and a second bacterial strain comprising aquatic Pseudomonas spp. with a 16S nucleic acid sequence that is at least about 97% identical to any one of the Pseudomonas spp. listed in Table 1 or Table 2; and (b) growing the plant element comprising the composition comprising the first bacterial strain and the second bacterial strain into the plant, wherein the plant grown from the contacted plant element has an improvement in the trait of agronomic importance as compared to a plant not contacted with the composition.

2. A method of improving a trait of agronomic importance in a plant, the method comprising: (a) contacting a plant element of the plant with a composition comprising one or more of the microbes listed in Table 1, Table 2, Table 3 or Table 4; and (b) growing the plant element comprising the composition comprising one or more of the microbes listed in Table 1, Table 2, Table 3 or Table 4 into the plant, wherein the plant grown from the contacted plant element has an improvement in the trait of agronomic importance as compared to a plant not contacted with the composition.

3. The method of claim 1 or claim 2, wherein the trait of agronomic importance is selected from the group consisting of: germination rate, emergence rate, shoot biomass, root biomass, root length, number of lateral roots, root surface area, seedling root length, seedling shoot length, leaf surface area, sugar holding capacity, sugar content and yield.

4. The method of claim 2, wherein the trait of agronomic importance is sugar holding capacity or sugar content, and wherein the plant is a grape or a melon.

5. A method of improving a trait of agronomic importance in a plant, the method comprising: growing the plant from a plant reproductive element that has been contacted with a composition comprising a first bacterial strain comprising Clostridium spp. with a 16S nucleic acid sequence that is at least about 97% identical to any one of the Clostridium spp. listed in Table 1 or Table 2 and a second bacterial strain comprising aquatic Pseudomonas spp. with a 16S nucleic acid sequence that is at least about 97% identical to any one of the Pseudomonas spp. listed in Table 1 or Table 2; wherein the plant grown from the contacted reproductive element comprises the composition comprising the first bacterial strain and the second bacterial strain, and wherein the plant grown from the contacted plant reproductive element has an improvement in a trait of agronomic importance as compared to an isoline plant grown from a plant reproductive element not contacted with the composition.

6. A method of improving a trait of agronomic importance in a plant, the method comprising growing the plant from a plant reproductive element that has been contacted with a composition comprising one or more of the microbes listed in Table 1, Table 2, Table 3 or Table 4; wherein the plant grown from the contacted reproductive element comprises the composition comprising the one or more microbes, and wherein the plant grown from the contacted plant reproductive element has an improvement in a trait of agronomic importance as compared to an isoline plant grown from a plant reproductive element not contacted with the composition.

7. The method of claim 5 or claim 6, wherein the trait of agronomic importance is selected from the group consisting of: germination rate, emergence rate, shoot biomass, root biomass, root length, number of lateral roots, root surface area, seedling root length, seedling shoot length, and yield.

8. A method of increasing the sugar content of a grape or melon, the method comprising: contacting an effective amount of a composition comprising a first bacterial strain comprising Clostridium spp. with a 16S nucleic acid sequence that is at least about 97% identical to any one of the Clostridium spp. listed in Table 1 or Table 2 and a second bacterial strain comprising aquatic Pseudomonas spp. with a 16S nucleic acid sequence that is at least about 97% identical to any one of the Pseudomonas spp. listed in Table 1 or Table 2 with an external surface of the grape or melon wherein the sugar content of the grape or melon contacted with the composition has an increased sugar content as compared to a plant not contacted with the composition.

9. A method of increasing the sugar content of a grape or melon, the method comprising: contacting an effective amount of a composition comprising one or more of the microbes listed in Table 1, Table 2, Table 3 or Table 4 with an external surface of the grape or melon wherein the sugar content of the grape or melon contacted with the composition has an increased sugar content as compared to a plant not contacted with the composition.

10. A method of increasing the sugar holding capacity of a grape or melon, the method comprising contacting an effective amount of a composition comprising a first bacterial strain comprising Clostridium spp. with a 16S nucleic acid sequence that is at least about 97% identical to any one of the Clostridium spp. listed in Table 1 or Table 2 and a second bacterial strain comprising aquatic Pseudomonas spp. with a 16S nucleic acid sequence that is at least about 97% identical to any one of the Pseudomonas spp. listed in Table 1 or Table 2 with an external surface of the grape or melon wherein the sugar holding capacity of the grape or melon the contacted with the composition has an increased sugar content as compared to a plant not contacted with the composition.

11. A method of increasing the sugar holding capacity of a grape or melon, the method comprising contacting an effective amount of a composition comprising one or more of the microbes listed in Table 1, Table 2, Table 3 or Table 4 with an external surface of the grape or melon wherein the sugar holding capacity of the grape or melon contacted with the composition has an increased sugar content as compared to a plant not contacted with the composition.

12. A method of increasing the amount of atmospheric derived nitrogen in a plant, the method comprising: exposing the plant to a composition comprising a first bacterial strain comprising Clostridium spp. with a 16S nucleic acid sequence that is at least about 97% identical to any one of the Clostridium spp. listed in Table 1 or Table 2 and a second bacterial strain comprising aquatic Pseudomonas spp. with a 16S nucleic acid sequence that is at least about 97% identical to any one of the Pseudomonas spp. listed in Table 1 or Table 2, and exposing the plant to one or more nitrogen-fixing bacteria thereby increasing an amount of atmospheric derived nitrogen within the plant that is exposed to the composition and the one or more nitrogen-fixing bacteria relative to a plant that is not exposed to the composition.

13. A method of increasing the amount of atmospheric derived nitrogen in a plant, the method comprising: exposing the plant to a composition comprising one or more of the microbes listed in Table 1, Table 2, Table 3 or Table 4, and exposing the plant to one or more nitrogen-fixing bacteria thereby increasing an amount of atmospheric derived nitrogen within the plant that is exposed to the composition and the one or more nitrogen-fixing bacteria relative to a plant that is not exposed to the composition.

14. The method of claim 12 or claim 13, further comprising exposing or contacting the plant with a nitrogen fixing bacteria.

15. A method of increasing nitrogen production in a plant, the method comprising: (a) contacting a plant element of the plant with a composition comprising a first bacterial strain comprising Clostridium spp. with a 16S nucleic acid sequence that is at least about 97% identical to any one of the Clostridium spp. listed in Table 1 or Table 2 and a second bacterial strain comprising aquatic Pseudomonas spp. with a 16S nucleic acid sequence that is at least about 97% identical to any one of the Pseudomonas spp. listed in Table 1 or Table 2 and (b) growing the plant element comprising the first bacterial strain and the second bacterial strain into the plant, wherein the plant grown from the contacted plant element produces more nitrogen as compared to a plant not contacted with the composition.

16. A method of increasing nitrogen production in a plant, the method comprising: (a) contacting a plant element of the plant with a composition comprising one or more of the microbes listed in Table 1, Table 2, Table 3 or Table 4 and (b) growing the plant element comprising the one or more of the microbes into the plant, wherein the plant grown from the contacted plant element produces more nitrogen as compared to a plant not contacted with the composition.
